(19) United States
(12) Patent Application Publication
(10) Pub. No.: US 2003/0225054 A1
(43) Pub. Date: Dec. 4, 2003

(54) Combined use of tace inhibitors and COX2 inhibitors as anti-inflammatory agents

(76) Inventor: Jingwu Duan, Yardley, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/453,036

(22) Filed: Jun. 3, 2003

Related U.S. Application Data
(60) Provisional application No. 60/385,656, filed on Jun. 3, 2002.

(51) Int. Cl. A61K 31/397; A61K 31/55; A61K 31/4747; A61K 31/445; A61K 31/4015; A61K 31/16; A61K 31/195

(52) U.S. Cl. 514/210.02; 514/212.02; 514/212.03; 514/327; 514/278; 514/409; 514/424; 514/563; 514/575

(57) ABSTRACT

This invention relates to a method of treating inflammatory diseases in a mammal comprising administering to the mammal a therapeutically effective amount of a combination of: (i) at least one TACE inhibitor of Formula (I), (II), (III), or (IV) of the present invention or a stereoisomer or pharmaceutically acceptable salt form thereof, (ii) one or more anti-inflammatory agents selected from the group consisting of: selective COX-2 inhibitors, interleukin-1 antagonists, dihydroorotate synthase inhibitors, p38 MAP kinase inhibitors, TNF-α inhibitors, TNF-α sequestration agents, and methotrexate. The invention also relates to compositions and kits containing the same.

### CROSS-REFERENCE TO RELATED APPLICATIONS

This application claims the priority benefits of U.S. Provisional Application No. 60/385,656, filed Jun. 3, 2002, which is fully incorporated herein by reference.

### FIELD OF THE INVENTION

This invention relates to a method of treating inflammatory diseases in a mammal comprising administering to the mammal a therapeutically effective amount of a combination of: (i) at least one TACE inhibitor of Formula (I), (II), (III), or (IV), and (ii) one or more anti-inflammatory agents selected from the group consisting of: selective COX-2 inhibitors, interleukin-1 antagonists, dihydroorotate synthase inhibitors, p38 MAP kinase inhibitors, TNF-α inhibitors, TNF-α sequestration agents, and methotrexate. The invention also relates to compositions and kits containing the same.

### BACKGROUND OF THE INVENTION

There is now a body of evidence that metalloproteases (MP) are important in the uncontrolled breakdown of connective tissue, including proteoglycan and collagen, leading to resorption of the extracellular matrix. This is a feature of many pathological conditions, such as rheumatoid and osteoarthritis, corneal, epidermal or gastric ulceration; tumor metastasis or invasion; periodontal disease and bone disease. Normally these catabolic enzymes are tightly regulated at the level of their synthesis as well as at their level of extracellular activity through the action of specific inhibitors, such as alpha-2-macroglobulins and TIMPs (tissue inhibitors of metalloprotease), which form inactive complexes with the MP's.

Osteo- and Rheumatoid Arthritis (OA and RA respectively) are destructive diseases of articular cartilage characterized by localized erosion of the cartilage surface. Findings have shown that articular cartilage from the femoral heads of patients with OA, for example, had a reduced incorporation of radiolabeled sulfate over controls, suggesting that there must be an enhanced rate of cartilage degradation in OA (Mankin et al. J. Bone Joint Surg. 1970, 52A, 424-434). There are four classes of protein degradative enzymes in mammalian cells: serine, cysteine, aspartic and metalloproteases. The available evidence supports that it is the metalloproteases that are responsible for the degradation of the extracellular matrix of articular cartilage in OA and RA. Increased activities of collagenases and stromelysin have been found in OA cartilage and the activity correlates with severity of the lesion (Mankin et al. Arthritis Rheum. 1978, 21, 761-766, Woessner et al. Arthritis Rheum. 1983, 26, 63-68 and Woessner et al. Arthritis Rheum. 1984, 27, 305-312). In addition, aggrecanase has been identified as providing the specific cleavage product of proteoglycan found in RA and OA patients (Lohmander L. S. et al. Arthritis Rheum. 1993, 36, 1214-22).

Therefore, metalloproteases (MP) have been implicated as the key enzymes in the destruction of mammalian cartilage and bone. It can be expected that the pathogenesis of such diseases can be modified in a beneficial manner by the administration of MP inhibitors, and many compounds have been suggested for this purpose (see Wahi et al. Ann. Rep. Med. Chem. 1990, 25, 175-184, AP, San Diego).

Tumor necrosis factor-α (TNF-α) is a cell-associated cytokine that is processed from a 26 kd precursor form to a 17 kd active form. TNF-α has been shown to be a primary mediator in humans and in animals, of inflammation, fever, and acute phase responses, similar to those observed during acute infection and shock. Excess TNF-α has been shown to be lethal. There is now considerable evidence that blocking the effects of TNF-α with specific antibodies can be beneficial in a variety of circumstances including autoimmune diseases such as rheumatoid arthritis (Feldman et al. Lancet 1994, 344, 1105), non-insulin dependent diabetes mellitus (Lohmander, L. S. et al. Arthritis Rheum. 1993, 36, 1214-22) and Crohn's disease (MacDonald et al. Clin. Exp. Immunol. 1990, 81, 301).

Compounds which inhibit the production of TNF-α are therefore of therapeutic importance for the treatment of inflammatory disorders. Recently, TNF-α converting enzyme (TACE), the enzyme responsible for TNF-α release from cells, were purified and sequenced (Black et al. Nature 1997, 385, 729; Moss et al. Nature 1997, 385, 733). This invention describes molecules that inhibit this enzyme and hence the secretion of active TNF-α from cells. These novel molecules provide a means of mechanism based therapeutic intervention for diseases including but not restricted to septic shock, haemodynamic shock, sepsis syndrome, post ischemic reperfusion injury, malaria, Crohn's disease, inflammatory bowel diseases, mycobacterial infection, meningitis, psoriasis, congestive heart failure, fibrotic diseases, cachexia, graft rejection, cancer, diseases involving angiogenesis, autoimmune diseases, skin inflammatory diseases, OA, RA, multiple sclerosis, radiation damage, hyperoxic alveolar injury, periodontal disease, HIV and non-insulin dependent diabetes melitus.

Since excessive TNF-α production has been noted in several disease conditions also characterized by MMP-mediated tissue degradation, compounds which inhibit both MMPs and TNF-α production may also have a particular advantage in diseases where both mechanisms are involved.

Prostaglandins (PG) play a major role in the inflammation process and the inhibition of PG production has been a common target of anti-inflammatory drug discovery. Many NSAIDS have been found to prevent the production of PG by inhibiting the enzyme cyclooxygenase (COX). Among the two isoforms of COXs, COX-1 is constitutively expressed. COX-2 is an inducible isozyme associated with inflammation. Selective COX-2 inhibitor was believed to maintain the efficacy of traditional NSAIDs, which inhibit both isozymes, and produce fewer and less drastic side effects. As a result, development of selective COX-2 inhibitors has attracted major interest in the pharmaceutical industry. Because of the significant roles of PGs and TNF-α in inflammation, combined use of COX-2 and TACE inhibitors may have superior efficacy to either therapy alone in some inflammatory diseases.

Compounds which selectively inhibit MPs, in particular aggrecanase and TNF-α have been described, for example, in U.S. Pat. Nos. 6,057,336, 6,268,379, 6,365,587, 6,376,665, WO99/41246, WO99/65867, WO00/59285, WO01/70673, WO01/70734, WO02/04416, and WO 02/28846. It has been shown that these compounds inhibit this conversion and hence the secretion of active TNF-α from cells.

Compounds which selectively inhibit cyclooxygenase-2 have been described, for example, in U.S. Pat. Nos. 5,380,738; 5,344,991; 5,393,790; 5,466,823; 5,434,178; 5,474,995 and 5,510,368; and WO documents WO 96/06840; WO 96/03388; WO 96/03387; WO 95/15316; WO 94/15932; WO 94/27980; WO 95/00501; WO 94/13635; WO 94/20480 and WO 94/26731.

WO 01/00229 describes combinations of TNF antagonists and COX-2 inhibitors for the treatment of inflammation.

None of the above references teaches or suggests the method of treating inflammatory diseases comprising administering to the mammal a therapeutically effective amount of a combination use of the compounds of the present invention that is described in detail below.

### SUMMARY OF THE INVENTION

The present invention provides a method of inflammatory diseases in a mammal comprising administering to the mammal a therapeutically effective amount of a combination of: (i) at least one TACE inhibitor, said TACE inhibitor being a compound of Formula (I), (II), (III), or (IV) (shown below), and (ii) one or more anti-inflammatory agents selected from the group consisting of: selective COX-2 inhibitors, interleukin-1 antagonists, dihydroorotate synthase inhibitors, p38 MAP kinase inhibitors, TNF-α inhibitors, TNF-α sequestration agents, and methotrexate.

In the present invention, it has been discovered that the administration of a TACE inhibitor of Formula (I), (II), (III), or (IV) (component (i)) in combination with a selective cycooxygenase-2 inhibitor (component (ii)), not only result in reduction of inflammation in patients suffering from inflammatory disease, but also maintain and/or increase the range of motion of joints in patients suffering from arthritic disease. The methods, combinations of the present invention provide effective therapy for treating inflammatory and arthritic diseases, for example, rheumatoid arthritis, with reduced adverse side effects as compared to such methods known in the art. The invention also relates to pharmaceutical compositions and kits containing the same.

### DETAILED DESCRIPTION OF PREFERRED EMBODIMENTS

The present invention provides a method of treating inflammatory diseases, such as rheumatoid arthritis, osteoarthritis and Crohn's disease, in a mammal comprising administering to the mammal a therapeutically effective amount of a combination of: (i) least one TACE inhibitor, said TACE inhibitor being a compound of Formula (I), (II), (III), or (IV) (shown below), and (ii) one or more anti-inflammatory agents selected from the group consisting of: selective COX-2 inhibitors, interleukin-1 antagonists, dihydroorotate synthase inhibitors, p38 MAP kinase inhibitors, TNF-α inhibitors, TNF-α sequestration agents, and methotrexate.

The methods, combinations and compositions of the present invention can be useful for the treatment or prevention of diseases in a mammal including, but not limited to, diseases such as: acute infection, acute phase response, age related macular degeneration, alcoholic liver disease, allergy, allergic asthma, anorexia, aneurism, aortic aneurism, asthma, atherosclerosis, atopic dermatitis, autoimmune disease, autoimmune hepatitis, Bechet's disease, cachexia, calcium pyrophosphate dihydrate deposition disease, cardiovascular effects, chronic fatigue syndrome, chronic obstruction pulmonary disease, coagulation, congestive heart failure, corneal ulceration, Crohn's disease, enteropathic arthropathy, Felty's syndrome, fever, fibromyalgia syndrome, fibrotic disease, gingivitis, glucocorticoid withdrawal syndrome, gout, graft versus host disease, hemorrhage, HIV infection, hyperoxic alveolar injury, infectious arthritis, inflammation, intermittent hydrarthrosis, Lyme disease, meningitis, multiple sclerosis, myasthenia gravis, mycobacterial infection, neovascular glaucoma, osteoarthritis, pelvic inflammatory disease, periodontitis, polymyositis/dermatomyositis, post-ischaemic reperfusion injury, post-radiation asthenia, psoriasis, psoriatic arthritis, pulmonary emphysema, pydoderma gangrenosum, relapsing polychondritis, Reiter's syndrome, rheumatic fever, rheumatoid arthritis, sarcoidosis, scleroderma, sepsis syndrome, Still's disease, shock, Sjogren's syndrome, skin inflammatory diseases, solid tumor growth and tumor invasion by secondary metastases, spondylitis, stroke, systemic lupus erythematosus, ulcerative colitis, uveitis, vasculitis, and Wegener's granulomatosis.

Said TACE inhibitors useful in the present invention are selected from compounds of Formula (I), (II), (III), or (IV):

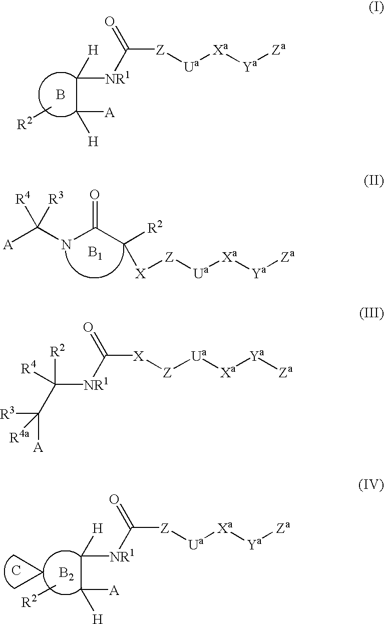

or a stereoisomer or pharmaceutically acceptable salt form thereof, wherein;

A, at each occurrence, is independently selected from —COR⁵, —CO₂H, CH₂CO₂H, —CONHOH, —CONHOR⁵, —CONHOR⁶, —N(OH)COR⁵, —SH, and —CH₂SH;

ring B is a 4-7 membered non-aromatic ring comprising: carbon atoms, 0-1 carbonyl groups, 0-1 double bonds, and from 0-2 ring heteroatoms selected from O, N, and NR² and substituted with 0-3 R^{c}; provided that ring B contains other than a O—O or N—O bond;

ring B₁ is a 4-7 membered cyclic amide containing from 0-2 additional heteroatoms selected from O, NR², and S(O)ₚ, and 0-1 additional carbonyl groups and 0-1 vinyl groups;

ring B₂ is a 4-7 membered non-aromatic carbocyclic or heterocyclic ring comprising: carbon atoms, 0-1 carbonyl groups, 0-1 double bonds, and from 0-2 ring heteroatoms selected from O, N, and NR², provided that ring B contains other than a O—O bond;

ring C forms a spiro ring on Ring B₂ and is a 4-10 membered carbocycle substituted with 0-3 R^{g} or a 4-10 membered heterocycle comprising: carbon atoms, 0-3 carbonyl groups, 0-4 double bonds, and from 0-4 ring heteroatoms selected from O, N, NR², and S(O)ₚ and substituted with 0-3 R^{g}, provided that ring C contains other than a S—S, O—O, or S—O bond;

X, at each occurrence, is absent or is independently selected from C₁₋₄ alkylene, C₂₋₄ alkenylene, C₂₋₄ alkynylene;

Z, at each occurrence, is absent or is independently selected from a C₃₋₆ carbocycle substituted with 0-4 R^{b} and a 5-6 membered heterocyclic system containing from 1-4 heteroatoms selected from the group consisting of N, O, and S and substituted with 0-5 R^{b};

U^{a}, at each occurrence, is absent or is independently selected from: O, NR^{a}, C(O), C(O)O, C(O)NR^{a}, NR^{a}C(O), S(O)ₚ, and S(O)ₚNR^{a};

X^{a}, at each occurrence, is absent or is independently selected from C₁₋₄ alkylene, C₂₋₄ alkenylene, and C₂₋₄ alkynylene;

Y^{a}, at each occurrence, is absent or is independently selected from O and NR^{a};

Z^{a}, at each occurrence, is absent or is independently selected from H, a C₃₋₁₀ carbocycle substituted with 0-5 R^{c} and a 5-10 membered heterocyclic system containing from 1-4 heteroatoms selected from the group comprising N, O, and S(O)ₚ and substituted with 0-5 R^{c};

provided that Z, U^{a}, Y^{a}, and Z^{a} do not combine to form a N—N, N—O, O—N, O—O, S(O)ₚ—O, O—S(O)ₚ or S(O)ₚ—S(O)ₚ group;

R¹, at each occurrence, is independently selected from H, C₁₋₄ alkyl, phenyl and benzyl;

R², at each occurrence, is independently selected from Q, C₁₋₆ alkylene-Q, C₂₋₆ alkenylene-Q, C₂₋₆ alkynylene-Q, —(CR^{a}R^{a1})ᵣ₁O(CR^{a}R^{a1})ᵣ-Q, —(CR^{a}R^{a1})ᵣ₁NR^{a}(CR^{a}R^{a1})ᵣ-Q, —(CR^{a}R^{a1})ᵣ₁C(O)(CR^{a}R^{a1})ᵣ-Q, —(CR^{a}R^{a1})ᵣ₁C(O)O(CR^{a}R^{a1})ᵣ-Q, —(CR^{a}R^{a1})ᵣC(O)NR^{a}R^{a1}, —(CR^{a}R^{a1})ᵣ₁C(O)NR^{a}(CR^{a}R^{a1})ᵣ-Q, —(CR^{a}R^{a1})ᵣ₁S(O)ₚ(CR^{a}R^{a1})ᵣ-Q, and —(CR^{a}R^{a1})ᵣ₁SO₂NR^{a}(CR^{a}R^{a1})ᵣ-Q;

R³, at each occurrence, is independently selected from H, C₁₋₆ alkylene-Q, C₂₋₆ alkenylene-Q, C₂₋₆ alkynylene-Q, —(CH₂)ᵣ₁O(CH₂)ᵣ-Q, (CH₂)ᵣ₁NR^{a}(CH₂)ᵣ-Q, —(CH₂)ᵣ₁C(O)(CH₂)ᵣ-Q, —(CH₂)ᵣ₁C(O)NR^{a}(CH₂)ᵣ-Q, —(CH₂)ᵣ₁NR^{a}C(O)(CH₂)ᵣ-Q, —(CH₂)ᵣ₁OC(O)NR^{a}(CH₂)ᵣ-Q, —(CH₂)ᵣ₁NR^{a}C(O)O(CH₂)ᵣ-Q, —(CH₂)ᵣ₁NR^{a}C(O)NR^{a}(CH₂)ᵣ-Q, —(CH₂)ᵣ₁S(O)ₚ(CH₂)ᵣ-Q, —(CH₂)ᵣ₁SO₂NR^{a}(CH₂)ᵣ-Q, —(CH₂)ᵣ₁NR^{a}SO₂(CH₂)ᵣ-Q, and —(CH₂)ᵣ₁NR^{a}SO₂NR^{a}(CH₂)ᵣ-Q;

Q, at each occurrence, is independently selected from H, a C₃₋₆ carbocycle substituted with 0-5 R^{d} and a 5-10 membered heterocyclic system containing from 1-4 heteroatoms selected from the group consisting of N, O, and S and substituted with 0-5 R^{d};

R⁴, at each occurrence, is independently selected from H and C₁₋₆ alkyl;

R^{4a} is H or C₁₋₆ alkyl;

alternatively, R³ and R⁴ in Formula (II) together with the carbon atom to which they are attached combine to form a 3-6 membered carbocyclic or heterocyclic ring comprising carbon atoms and 0-2 ring heteroatoms selected from O, N, NR^{c}, and S(O)ₚ and substituted with 0-1 R^{c};

alternatively, R¹ and R² in Formula (III) together with the carbon and nitrogen atoms to which they are attached combine to form a 3-10 membered heterocyclic ring comprising carbon atoms and, in addition to the nitrogen atom to which R¹ is attached, 0-1 ring heteroatoms selected from O, N, NR^{c}, and S(O)ₚ and substituted with 0-1 R^{c};

alternatively, R¹ and R³ in Formula (III) together with the carbon and nitrogen atoms to which they are attached combine to form a 4-6 membered heterocyclic ring comprising carbon atoms and, in addition to the nitrogen atom to which R¹ is attached, 0-1 ring heteroatoms selected from O, N, and NR^{c}, and substituted with 0-1 R^{c};

alternatively, R² and R⁴ in Formula (III) together with the carbon atom to which they are attached combine to form a 3-10 membered carbocyclic or heterocyclic ring comprising carbon atoms and 0-2 ring heteroatoms selected from O, N, NR^{c}, and S(O)ₚ and substituted with 0-3 R^{c};

alternatively, R³ and R^{4a} in Formula (III) together with the carbon atom to which they are attached combine to form a 3-6 membered carbocyclic or heterocyclic ring comprising carbon atoms and 0-2 ring heteroatoms selected from O, N, NR^{c}, and S(O)ₚ and substituted with 0-1 R^{e};

R⁵, at each occurrence, is selected from C₁₋₆ alkyl substituted with 0-2 R^{b}, and C₁₋₄ alkyl substituted with 0-2 R^{e};

R⁶, at each occurrence, is selected from phenyl, naphthyl, C₁₋₁₀ alkyl-phenyl-C₁₋₆ alkyl-, C₃₋₁₁ cycloalkyl, C₁₋₆ alkylcarbonyloxy-C₁₋₃ alkyl-, C₁₋₆ alkoxycarbonyloxy-C₁₋₃ alkyl-, C₂₋₁₀ alkoxycarbonyl, C₃₋₆ cycloalkylcarbonyloxy-C₁₋₃ alkyl-, C₃₋₆ cycloalkoxycarbonyloxy-C₁₋₃ alkyl-, C₃₋₆ cycloalkoxycarbonyl, phenoxycarbonyl, phenyloxycarbonyloxy-C₁₋₃ alkyl-, phenylcarbonyloxy-C₁₋₃ alkyl-, C₁₋₆ alkoxy-C₁₋₆ alkylcarbonyloxy-C₁₋₃ alkyl-, [5-(C₁-C₅ alkyl)-1,3-dioxa-cyclopenten-2-one-yl]methyl, [5-(R^{a})-1,3-dioxa-cyclopenten-2-one-yl]methyl, (5-aryl-1,3-dioxa-cyclopenten-2-one-yl)methyl, —C₁₋₁₀ alkyl-NR⁷R^{7a}, —CH(R⁸)OC(═O)R⁹, and —CH(R⁸)OC(═O)OR⁹;

R⁷, at each occurrence, is independently selected from H and C₁₋₆ alkyl, C₂₋₆ alkenyl, C₃₋₆ cycloalkyl-C₁₋₃ alkyl-, and phenyl-C₁₋₆ alkyl-;

R^{7a}, at each occurrence, is independently selected from H and C₁₋₆ alkyl, C₂₋₆ alkenyl, C₃₋₆ cycloalkyl-C₁₋₃ alkyl-, and phenyl-C₁₋₆ alkyl-;

R⁸, at each occurrence, is independently selected from H and C₁₋₄ linear alkyl;

R⁹, at each occurrence, is independently selected from H, C₁₋₆ alkyl substituted with 1-2 R^{f}, C₃₋₆ cycloalkyl substituted with 1-2 R^{f}, and phenyl substituted with 0-2 R^{b};

R^{a}, at each occurrence, is independently selected from H, C₁₋₄ alkyl, phenyl and benzyl;

R^{a1}, at each occurrence, is independently selected from H and C₁₋₄ alkyl;

alternatively, R^{a} and R^{a1} when attached to a nitrogen are taken together with the nitrogen to which they are attached to form a 5 or 6 membered ring comprising carbon atoms and from 0-1 additional heteroatoms selected from the group consisting of N, O, and S;

R^{a2}, at each occurrence, is independently selected from C₁₋₄ alkyl, phenyl, and benzyl;

R^{b}, at each occurrence, is independently selected from C₁₋₆ alkyl, OR^{a}, Cl, F, Br, ═O, —CN, NR^{a}R^{a1}, C(O)R^{a}, C(O)OR^{a}, C(O)NR^{a}R^{a1}, S(O)₂NR^{a}R^{a1}, S(O)ₚR^{a1}, and CF₃;

R^{c}, at each occurrence, is independently selected from C₁₋₆ alkyl, OR^{a}, Cl, F, Br, ═O, —CN, NR^{a}R^{a1}, C(O)R^{a}, C(O)OR^{a}, C(O)NR^{a}R^{a1}, S(O)₂NR^{a}R^{a1}, S(O)ₚR^{a1}, CF₃, (CH₂)ᵣ—C₃₋₆ carbocycle and a (CH₂)ᵣ-5-6 membered heterocycle comprising: carbon atoms and 1-4 heteroatoms selected from the group consisting of N, O, and S;

alternatively, two R^{c}s on the same carbon atom are taken together with the carbon atom to which they are attached to form a 5 or 6 membered ring comprising carbon atoms and from 0-1 additional heteroatoms selected from the group consisting of N, O, and S;

R^{c1}, at each occurrence, is independently selected from C₁₋₆ alkyl, OR^{a}, Cl, F, Br, I, ═O, —CN, NO₂, NR^{a}R^{a1}, C(O)R^{a}, C(O)OR^{a}, C(O)NR^{a}R^{a1}, R^{a}NC(O)NR^{a}R^{a1}, OC(O)NR^{a}R^{a1}, R^{a}NC(O)OR^{a}, S(O)₂NR^{a}R^{a1}, NR^{a}S(O)₂R^{a2}, NR^{a}S(O)₂NR^{a}R^{a1}, OS(O)₂NR^{a}R^{a1}, NR^{a}S(O)₂R^{a2}, S(O)ₚR^{a2}, CF₃, CF₂CF₃, CH₂F, and CHF₂;

R^{d}, at each occurrence, is independently selected from C₁₋₆ alkyl, OR^{a}, Cl, F, Br, ═O, —CN, NR^{a}R^{a1}, C(O)R^{a}, C(O)OR^{a}, C(O)NR^{a}R^{a1}, S(O)₂NR^{a}R^{a1}, S(O)ₚR^{a2}, CF₃, C₃₋₆ carbocyclic residue and a 5-6 membered heterocycle comprising: carbon atoms and 1-4 heteroatoms selected from the group consisting of N, O, and S;

R^{e}, at each occurrence, is selected from phenyl substituted with 0-2 R^{b} and biphenyl substituted with 0-2 R^{b};

R^{f}, at each occurrence, is selected from C₁₋₄ alkyl, C₃₋₆ cycloalkyl, C₁₋₅ alkoxy, phenyl substituted with 0-2 R^{b};

R^{g}, at each occurrence, is independently selected from C₁₋₆ alkyl, OR^{a}, Cl, F, Br, I, ═O, —CN, NO₂, NR^{a}R^{a1}, C(O)R^{a}, C(O)OR^{a}, C(O)NR^{a}R^{a1}, R^{a}NC(O)NR^{a}R^{a1}, OC(O)NR^{a}R^{a1}, R^{a}NC(O)OR^{a}, S(O)₂NR^{a}R^{a1}, NR^{a}S(O)₂R^{a2}, NR^{a}S(O)₂NR^{a}R^{a1}, OS(O)₂NR^{a}R^{a1}, NR^{a}S(O)₂R^{a2}, S(O)ₚR^{a2}, CF₃, CF₂CF₃, C₃₋₁₀ carbocycle substituted with 0-2 R^{c1}, (CR^{a}R^{a1})ᵣ₁—C₃₋₁₀ carbocycle substituted with 0-2 R^{c1}, a 5-14 membered heterocycle comprising carbon atoms and 1-4 heteroatoms selected from the group consisting of N, O, and S(O)ₚ and substituted with 0-2 R^{c1}, and (CR^{a}R^{a1})ᵣ₁-5-14 membered heterocycle comprising carbon atoms and 1-4 heteroatoms selected from the group consisting of N, O, and S(O)ₚ and substituted with 0-2 R^{c1};

p, at each occurrence, is selected from 0, 1, and 2;

r, at each occurrence, is selected from 0, 1, 2, 3, and 4; and

r1, at each occurrence, is selected from 0, 1, 2, 3, and 4.

Preferred compounds of component (i) useful in the method of the present invention are compounds selected from:

[1(R)]-3-amino-N-hydroxy-α-(2-methylpropyl)-2-oxo-3-[4-(2-quinolinylmethoxy)phenyl]-1-pyrrolidineacetamide;

[1(R)]-3-amino-N-hydroxy-α-(2-methylpropyl)-2-oxo-3-[4-(4-quinolinylmethoxy)phenyl]-1-pyrrolidineacetamide;

[1(R)]-3-amino-N-hydroxy-3-[4-[(2-methoxy-4-quinolinyl)methoxy]phenyl]-α-(2-methylpropyl)-2-oxo-1-pyrrolidineacetamide;

[1(R)]-3-amino-N-hydroxy-α-(2-methylpropyl)-2-oxo-3-[4-[(2-phenyl-4-quinolinyl)methoxy]phenyl]-1-pyrrolidineacetamide;

[1(R)]-3-amino-3-[4-[(2,6-dimethyl-4-quinolinyl)methoxy]phenyl]-N-hydroxy-α-(2-methylpropyl)-2-oxo-1-pyrrolidineacetamide;

[1(R)]-3-amino-3-[4-[(2-chloro-4-quinolinyl)methoxy]phenyl]-N-hydroxy-α-(2-methylpropyl)-2-oxo-1-pyrrolidineacetamide;

[1(R)]-3-amino-N-hydroxy-a-(2-methylpropyl)-3-[4-[(2-methyl-4-quinolinyl)methoxy]phenyl]-2-oxo-1-pyrrolidineacetamide;

[1(R)]-3-amino-3-[4-[(2-chloro-4-quinolinyl)methoxy]phenyl]-N-hydroxy-α-[2-(methylsulfonyl)ethyl]-2-oxo-1-pyrrolidineacetamide;

[1(R)]-3-amino-N-hydroxy-3-[4-[(2-methyl-4-quinolinyl)methoxy]phenyl]-α-[2-(methylsulfonyl)ethyl]-2-oxo-1-pyrrolidineacetamide;

[1(R)]-3-amino-N-hydroxy-3-[4-[(2-methoxy-4-quinolinyl)methoxy]phenyl]-α-[2-(methylsulfonyl)ethyl]-2-oxo-1-pyrrolidineacetamide;

[1(R)]-N-hydroxy-3-(methylamino)-α-(2-methylpropyl)-3-[4-[(2-methyl-4-quinolinyl)methoxy]phenyl]-2-oxo-1-pyrrolidineacetamide;

[1(R)]-α-[3-amino-2-oxo-3-[4-(4-quinolinylmethoxy)phenyl]-1-pyrrolidinyl]-N-hydroxy-4-piperidineacetamide;

[1(R)]-3-amino-N-hydroxy-α-(1-methylethyl)-2-oxo-3-[4-(4-quinolinylmethoxy)phenyl]-1-pyrrolidineacetamide;

[1(R)]-3-amino-α-cyclohexyl-N-hydroxy-2-oxo-3-[4-(4-quinolinylmethoxy)phenyl]-1-pyrrolidineacetamide;

[1(R)]-3-amino-α-(1,1-dimethylethyl)-N-hydroxy-2-oxo-3-[4-(4-quinolinylmethoxy)phenyl]-1-pyrrolidineacetamide;

[1(R)]-3-amino-α-(1,1-dimethylethyl)-N-hydroxy-2-oxo-3-[4-[(2-methyl-4-quinolinyl)methoxy]phenyl]-1-pyrrolidineacetamide;

[1(R)]-3-amino-N-hydroxy—(1-methylethyl)-2-oxo-3-[4-[(2-methyl-4-quinolinyl)methoxy]phenyl]-1-pyrrolidineacetamide;

[1(R)]-3-amino-N-hydroxy-α-(1-methylethyl)-2-oxo-3-[4-[(2,6-dimethyl-4-quinolinyl)methoxy]phenyl]-1-pyrrolidineacetamide;

(3S,4S)-N-hydroxy-1-isopropyl-4-({4-[(2-methyl-4-quinolinyl)methoxy]benzoyl}amino)-3-pyrrolidinecarboxamide;

(3S,4S)-N-hydroxy-4-({4-[(2-methyl-4-quinolinyl)methoxy]benzoyl}amino)-1-(2-propynyl)-3-pyrrolidinecarboxamide;

(3S,4S)-1-(2-butynyl)-N-hydroxy-4-({4-[(2-methyl-4-quinolinyl)methoxy]benzoyl}amino)-3-pyrrolidinecarboxamide;

(3R,4S)-N-hydroxy-4-({4-[(2-methyl-4-quinolinyl)methoxy]benzoyl}amino)tetrahydro-3-furancarboxamide;

(3S,4S)-4-{[4-(2-butynyloxy)benzoyl]amino}-N-hydroxy-1-isopropyl-3-pyrrolidinecarboxamide;

(1S,2R)-N-hydroxy-2-[[[4-[(2-methyl-4-quinolinyl)methoxy]phenyl]carbonyl]amino]-1-cyclopentanecarboxamide;

(3S,4R)-N-hydroxy-1-methyl-4-[[[4-[(2-methyl-4-quinolinyl)methoxy]phenyl]carbonyl]amino]-3-piperidinecarboxamide;

(3S,4S)-N-hydroxy-1-(1-methylethyl)-3-[[[4-[(2-methyl-4-quinolinyl)methoxy]phenyl]carbonyl]amino]-4-piperidinecarboxamide;

(3R,4R)-N-hydroxy-4-({4-[(2-methyl-4-quinolinyl)methoxy]benzoyl}amino)tetrahydro-2H-pyran-3-carboxamide;

(3S,4S)-1-tert-butyl-N-hydroxy-3-({4-[(2-methyl-4-quinolinyl)methoxy]benzoyl}amino)-4-piperidinecarboxamide;

(3S,4S)-3-({4-[(2,5-dimethylbenzyl)oxy]benzoyl}amino)-N-hydroxy-4-piperidinecarboxamide;

N-{4-[2-(hydroxyamino)-2-oxoethyl]-4-piperidinyl}-4-[(2-methyl-4-quinolinyl)methoxy]benzamide;

N-[3-[2-(hydroxyamino)-2-oxoethyl]-1-(4-pyridinylmethyl)-3-piperidinyl]-4-[(2-methyl-4-quinolinyl)methoxy]benzamide;

N-{4α-[2-(hydroxyamino)-2-oxoethyl]-2β,6β-dimethyl-4-piperidinyl}-4-[(2-methyl-4-quinolinyl)methoxy]benzamide;

N-{4-[2-(hydroxyamino)-2-oxoethyl]hexahydro-1H-azepin-4-yl}-4-[(2-methyl-4-quinolinyl)methoxy]benzamide;

N-{4-[2-(hydroxyamino)-2-oxoethyl]tetrahydro-2H-pyran-4-yl}-4-[(2-methyl-4-quinolinyl)methoxy]benzamide;

N-{(3R)-3-[2-(hydroxyamino)-2-oxoethyl]tetrahydro-2H-pyran-3-yl}-4-[(2-methyl-4-quinolinyl)methoxy]benzamide;

N-{(3S)-3-[2-(hydroxyamino)-2-oxoethyl]tetrahydro-2H-pyran-3-yl}-4-[(2-methyl-4-quinolinyl)methoxy]benzamide;

N-{4-[2-(hydroxyamino)-2-oxoethyl]tetrahydro-2H-thiopyran-4-yl}-4-[(2-methyl-4-quinolinyl)methoxy]benzamide;

N-{4-[2-(hydroxyamino)-2-oxoethyl]-1, 1-dioxidotetrahydro-2H-thiopyran-4-yl}-4-[(2-methyl-4-quinolinyl)methoxy]benzamide;

N-{3-[2-(hydroxyamino)-2-oxoethyl]tetrahydro-3-furanyl}-4-[(2-methyl-4-quinolinyl)methoxy]benzamide;

(5R,7S,8R)-N-hydroxy-8-({4-[(2-methyl-4-quinolinyl)methoxy]benzoyl}amino)-1-oxaspiro[4.4]nonane-7-carboxamide;

(5S,7S,8R)-N-hydroxy-8-({4-[(2-methyl-4-quinolinyl)methoxy]benzoyl}amino)-1-oxaspiro[4.4]nonane-7-carboxamide;

(5R,7S,8R)-8-{[4-(2-butynyloxy)benzoyl]amino}-N-hydroxy-1-oxaspiro[4.4]nonane-7-carboxamide;

(5R,7S,8R)-N-hydroxy-8-({4-[(2-isopropyl-1H-benzimidazol-1-yl)methyl]benzoyl}amino)-1-oxaspiro[4.4]nonane-7-carboxamide;

(5R,7S,8R)-N-hydroxy-8-[(4-{[2-(trifluoromethyl)-1H-benzimidazol-1-yl]methyl}benzoyl)amino]-1-oxaspiro[4.4]nonane-7-carboxamide;

(5R,7S,8R)-8-[(4-{[2-(1, 1-difluoroethyl)-1H-benzimidazol-1-yl]methyl}benzoyl)amino]-N-hydroxy-1-oxaspiro[4.4]nonane-7-carboxamide;

(5R,7S,8R)-8-({4-[(3,5-dimethyl-1H-pyrazol-4-yl)methyl]benzoyl}amino)-N-hydroxy-1-oxaspiro[4.4]nonane-7-carboxamide;

(5R,7S,8R)-N-hydroxy-8-({4-[(2-methyl-4-quinolinyl)methyl]benzoyl}amino)-1-oxaspiro[4.4]nonane-7-carboxamide;

(5R,7S,8R)-N-hydroxy-8-[(4-{[2-(trifluoromethyl)-4-quinolinyl]methyl}benzoyl)amino]-1-oxaspiro[4.4]nonane-7-carboxamide; and

(5R,7S,8R)-N-hydroxy-8-({4-[(2-isopropyl-4-quinolinyl)methyl]benzoyl}amino)-1-oxaspiro[4.4]nonane-7-carboxamide;

or a pharmaceutically acceptable salt form thereof.

In one embodiment, the component (ii) in the present invention is a selective COX-2 inhibitor. For example, the compounds have a selectivity ratio of cyclooxygenase-2 inhibition over cyclooxygenase-1 inhibition of at least 50, and in another embodiment have a selectivity ratio of at least 100. Such selectivity ratios may indicate an ability to reduce the incidence of common NSAID-induced side effects.

Nonlimiting examples of cyclooxygenase-2 inhibitors that may be used in the present invention include compounds or pharmaceutically acceptable salts, as follows:

4-[4-(methyl-sulfonyl)phenyl]-3-phenyl-2(5H)-furanone (MK-966, Vioxx®; rofecoxib);

4-(5-(4-methylphenyl)-3-trifluoromethyl)-1H-pyrazol-1-yl)-benzenesulfonamide (celecoxib; Celebrex®);

6-chloro-4-hydroxy-2-methyl-N-2-pyridinyl-2H-thieno[2,3-e]-1,2-thiazine-3-carboxamide-1,1-dioxide (lornoxicam, Safem®);

4-hydroxy-2-methyl-N-(5-methyl-2-thiazolyl)-1,1-dioxide-2H-1,2-benzothiazine-3-carboxamide (meloxicam);

N-(4-nitro-2-phenoxyphenyl)methanesulfonamide (nimesulide);

4-(5-methyl-3-phenyl-4-isoxazolyl)-benzenesulfonamide (valdecoxib);

2-(6-methylpyrid-3-yl)-3-(4-methylsulfinylphenyl)-5-chloropyridine (MK-663; L-791456, etoricoxib);

N-[[4-(5-methyl-3-phenyl-4-isoxazolyl)phenyllsulfonyl]-propanamide (parecoxib);

5,5-dimethyl-3-(1-methylethoxy)-4-[4-(methylsulfonyl)phenyl]-2(5H)-furanone (DFP);

N-[6-(2,4-difluorophenoxy)-2,3-dihydro-1-oxo-1H-inden-5-yl]

Methanesulfonamide (flosulide);

5-methyl-N-[4-(trifluoromethyl)phenyl]-4-isoxazolecarboxamide (leflunomide);

2-fluoro-α-methyl-4-(nitrooxy)butyl ester-[1,1′-biphenyl]-4-acetic acid (nitroflurbiprofen, HCT-1026);

1-(4-chlorobenzoyl)-3-[4-(4-fluorophenyl)thiazol-2-ylmethyl]-5-methoxy-2-methylindole (A-183827.0);

4-(4-cyclohexyl-2-methyloxazol-5-yl)-2-fluorobenzenesulfonamide (JTE-522);

3-(3-fluorophenyl)-5,5-dimethyl-4-[4-(methylsulfonyl)phenyl]-2(5H)-furanone (DFU);

3-(3,4-difluorophenoxy)-5-methyl-4-[4-(methylsulfonyl)phenyl]-5-(2,2,2-trifluoroethyl-2(5H)-Furanone (L-784512);

5,5-dimethyl-4-[4-(methylsulfonyl)phenyl]-3-phenoxy-2(5H)-furanone (L-783003);

3-[(5-bromo-2-pyridinyl)oxy]-5,5-dimethyl-4-[4-(methylsulfonyl)phenyl]-2(5H)-furanone (L-778736);

3-[4-(methylsulfonyl)phenyl]-2-(3,5-difluorophenyl)-2-cyclopenten-1-one (L-776967);

5-[4-(methylsulfonyl)-phenyl]-6-phenyl-thiazolo[3,2-b] [1,2,4]triazole (L-768277);

(S)-2-methyl-1-[(4-bromophenyl)methyl]-5-methoxy-β-1H-Indole-3-butanoic acid (L-761066);

N-[6-[(4-ethyl-2-thiazolyl)thio]-1,3-dihydro-1-oxo-5-isobenzofuranyl]methanesulfonamide (L-758115);

5-methoxy-2-methyl-1-(2,4,6-trichlorobenzoyl)-1H-indole-3-acetic acid (L-748780);

4-[2-(4-fluorophenyl)-3-thienyl]-benzenesulfonamide (L-746483);

N-[6-[(2,4-difluorophenyl)thio]-2,3-dihydro-1-oxo-1H-inden-5-yl]-methanesulfonamide (L-745337);

5-[[3,5-bis(1,1-dimethylethyl)-4-hydroxyphenyl]methylene]-2,4-thiazolidinedione (2-(O-methyloxime), PD-138387);

5-(4-fluorophenyl)-6-[4-(methylsulfonyl)phenyl]-spiro[2.4]hept-5-ene (SC-58451);

5-(4-fluorophenyl)-1-[4-(methylsulfonyl)phenyl]-3-(trifluoromethyl)-1H-pyrazole (SC-58231);

1-fluoro-4-[2-[4-(methylsulfonyl)phenyl]cyclopenten-1-yl] benzene (SC-57666);

4-[5-(4-chlorophenyl)-3-(difluoromethyl)-1H-pyrazol-1-yl]benzenesulfonaniide (SC-236);

N-[5-(4-fluoro)phenoxylthlophene-2-methanesulfonamide (RWJ-63556);

6-[[5-(4-chlorobenzoyl)-1,4-dimethyl-1H-pyrrol-2-yl]methyl]-3(2H)-pyridazinone (RS-57067-000);

5(E)-(3,5-di-tert-butyl-4-hydroxy)benzylidene-2-ethyl-1,2-isothiazolidine-1,1-dioxide (S-2474);

3-formylamino-7-methylsulfonylamino-6-phenoxy-4H-1-benzopyran-4-one (T-614);

N-[2-(cyclohexyloxy)-4-nitrophenyl]methanesulfonamide (NS-398);

AF-3161; AF-3162; COX-189; CS 502; CS-179; CT-3; GR-253035; HN-56249; LAS-33815; LAS-33826; LAS-34475; PD-164387; PD-138768; PD-098120; SC-906; SC-58236; S-2474; S-33516; SVT-2016, TP-72; UR-8813; UR-8877; UP-454-21; and UR-8880.

In another embodiment, the present invention provides a novel method of treating a disease or condition using a compound of Formula (II) or a stereoisomer or pharmaceutically acceptable salt form thereof, wherein the disease or condition is selected from age related macular degeneration, allergy, allergic asthma, aneurism, aortic aneurism, asthma, atherosclerosis, atopic dermatitis, autoimmune hepatitis, Bechet's disease, calcium pyrophosphate dihydrate deposition disease, chronic fatigue syndrome, chronic obstruction pulmonary disease, congestive heart failure, Crohn's disease, enteropathic arthropathy, Felty's syndrome, fibromyalgia syndrome, fibrotic disease, glucocorticoid withdrawal syndrome, gout, hyperoxic alveolar injury, infectious arthritis, inflammation, intermittent hydrarthrosis, Lyme disease, meningitis, myasthenia gravis, mycobacterial infection, pelvic inflammatory disease, polymyositis/dermatomyositis, post-ischaemic reperfusion injury, post-radiation asthenia, psoriatic arthritis, pulmonary emphysema, pydoderma gangrenosum, relapsing polychondritis, Reiter's syndrome, rheumatic fever, sarcoidosis, scleroderma, sepsis syndrome, Still's disease, Sjogren's syndrome, skin inflammatory diseases, spondylitis, stroke, systemic lupus erythematosus, ulcerative colitis, uveitis, vasculitis, and Wegener's granulomatosis.

The present invention comprises a pharmaceutical composition comprising a therapeutically effective amount of a TACE compound of and a cyclooxygenase-2 inhibitor compound in association with at lease one pharmaceutically acceptable carrier, adjuvant or diluent.

The present invention also includes pharmaceutical kits useful, for example, in the treatment or prevention of osteoarthritis or rheumatoid arthritis, which comprise one or more containers containing a pharmaceutical composition comprising a therapeutically effective amount of a TACE compound of and a COX-2 inhibitor compound. Such kits may further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, may also be included in the kit.

“Biologically active,” as used throughout the specification as a characteristic of tumor necrosis factor receptor antagonizing agent, means, for example, that a particular molecule shares sufficient amino acid sequence similarity with the embodiments of the present invention disclosed herein to be capable of binding detectable quantities of tumor necrosis factor receptor, transmitting a tumor necrosis factor stimulus to a cell, for example, as a component of a hybrid receptor construct, or cross-reacting with anti-tumor necrosis factor receptor antibodies raised against tumor necrosis factor receptor from natural (i.e., nonrecombinant) sources.

In one embodiment of the present invention, the biologically active tumor necrosis factor receptor antagonizing agent within the scope of the present invention are capable of binding greater than 0.1 nmoles tumor necrosis factor per nmole receptor, and in another embodiment, are capable of binding greater than 0.5 nmole tumor necrosis factor per nmole receptor in standard binding assays (see U.S. Pat. No. 5,605,690).

The phrase “combination therapy” (or “co-therapy”) embraces the administration of a TACE inhibitor and one or more anti-inflammatory agents as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected).

“Combination therapy” generally is not intended to encompass the administration of two or more of these therapeutic agents as part of separate monotherapy regimens that incidentally and arbitrarily result in the combinations of the present invention.

“Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule or intravenous injection having a fixed ratio of each therapeutic agent or in multiple, single capsules or intravenous injections for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues.

The therapeutic agents can be administered by the same route or by different routes. For example, a first therapeutic agent of the combination selected may be administered by intravenous injection while the other therapeutic agents of the combination may be administered orally.

The phrase “therapeutically effective” is intended to qualify the amount of each agent that will achieve the goal of improvement in arthritic disease severity and the frequency of incidence over treatment of each agent by itself, while avoiding adverse side effects typically associated with alternative therapies.

A “therapeutic effect” relieves to some extent one or more of the symptoms of an arthritic or inflammatory disorder. In reference to the treatment of rheumatoid arthritis, a therapeutic effect refers to one or more of the following: 1) relieving or reducing to some extent one or more of the symptoms associated with the disorder, 2) relieving or reducing to some extent gastrointestinal upset, 3) relieving or reducing to some extent mucosal ulcerations, 4) relieving or reducing to some extent renal impairment, 5) relieving or reducing to some extent pulmonary toxicity, and/or 6) relieving or reducing the side effects associated with the administration of other antiarthritic agents, such as disease modifying antirheumatic drugs.

Besides being useful for human treatment, the method, combinations, agents and compositions of the present invention are also useful for treatment of mammals, including, but not limited to, horses, dogs, cats, rats, mice, sheep, pigs, etc.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Geometric isomers of double bonds such as olefins and C═N double bonds can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated. All processes used to prepare compounds of the present invention and intermediates made therein are considered to be part of the present invention.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring.

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

When any variable (e.g., R^{b}) occurs more than one time in any constituent or formula for a compound, its definition at each occurrence is independent of its definition at every other occurrence. Thus, for example, if a group is shown to be substituted with 0-2 R⁶, then said group may optionally be substituted with up to two R⁶ groups and R⁶ at each occurrence is selected independently from the definition of R⁶. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl” or “alkylene” is intended to include both branched and straight-chain saturated aliphatic hydrocarbon groups having the specified number of carbon atoms. C₁₋₁₀ alkyl (or alkylene), is intended to include C₁, C₂, C₃, C₄, C₅, C₆, C₇, C₈, C₉, and C₁₀ alkyl groups. Examples of alkyl include, but are not limited to, methyl, ethyl, n-propyl, i-propyl, n-butyl, s-butyl, t-butyl, n-pentyl, and s-pentyl. “Haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, and pentachloroethyl. “Alkoxy” represents an alkyl group as defined above with the indicated number of carbon atoms attached through an oxygen bridge. C₁₋₁₀ alkoxy, is intended to include C₁, C₂, C₃, C₄, C₅, C₆, C₇, C₈, C₉, and C₁₀ alkoxy groups. Examples of alkoxy include, but are not limited to, methoxy, ethoxy, n-propoxy, i-propoxy, n-butoxy, s-butoxy, t-butoxy, n-pentoxy, and s-pentoxy. “Cycloalkyl” is intended to include saturated ring groups, such as cyclopropyl, cyclobutyl, or cyclopentyl. C₃₋₇ cycloalkyl, is intended to include C₃, C₄, C₅, C₆, and C₇ cycloalkyl groups. “Alkenyl” or “alkenylene” is intended to include hydrocarbon chains of either a straight or branched configuration and one or more unsaturated carbon-carbon bonds which may occur in any stable point along the chain, such as ethenyl and propenyl. C₂₋₁₀ alkenyl (or alkenylene), is intended to include C₂, C₃, C₄, C₅, C₆, C₇, C₈, C₉, and C₁₀ alkenyl groups. “Alkynyl” or “alkynylene” is intended to include hydrocarbon chains of either a straight or branched configuration and one or more triple carbon-carbon bonds which may occur in any stable point along the chain, such as ethynyl and propynyl. C₂₋₁₀ alkynyl (or alkynylene), is intended to include C₂, C₃, C₄, C₅, C₆, C₇, C₈, C₉, and C₁₀ alkynyl groups.

“Halo” or “halogen” as used herein refers to fluoro, chloro, bromo, and iodo; and “counterion” is used to represent a small, negatively charged species such as chloride, bromide, hydroxide, acetate, and sulfate.

As used herein, “carbocycle” or “carbocyclic residue” is intended to mean any stable 3, 4, 5, 6, or 7-membered monocyclic or bicyclic or 7, 8, 9, 10, 11, 12, or 13-membered bicyclic or tricyclic, any of which may be saturated, partially unsaturated, or aromatic. Examples of such carbocycles include, but are not limited to, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl.

As used herein, the term “heterocycle” or “heterocyclic group” is intended to mean a stable 5, 6, or 7-membered monocyclic or bicyclic or 7, 8, 9, or 10-membered bicyclic heterocyclic ring which is saturated, partially unsaturated or unsaturated (aromatic), and which consists of carbon atoms and 1, 2, 3, or 4 heteroatoms independently selected from the group consisting of N, NH, O and S and including any bicyclic group in which any of the above-defined heterocyclic rings is fused to a benzene ring. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. As used herein, the term “aromatic heterocyclic group” or “heteroaryl” is intended to mean a stable 5, 6, or 7-membered monocyclic or bicyclic or 7, 8, 9, or 10-membered bicyclic heterocyclic aromatic ring which consists of carbon atoms and 1, 2, 3, or 4 heteroatoms independently selected from the group consisting of N, NH, O and S. It is to be noted that total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles.

The term “independently selected from”, “independently, at each occurrence” or similar language, means that the labeled R substitution group may appear more than once and that each appearance may be a different atom or molecule found in the definition of that labeled R substitution group. Thus if the labeled R⁶ substitution group appear four times in a given permutation of Formula (I), then each of those labeled R⁶ substitution groups may be a different group falling in the definition of R⁶.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed, Mack Publishing Company, Easton, Pa., 1985, p. 1418, the disclosure of which is hereby incorporated by reference.

“Stable compound” and “stable structure” are meant to indicate a compound that is sufficiently robust to survive isolation to a useful degree of purity from a reaction mixture, and formulation into an efficacious therapeutic agent.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e, ═O) group, then 2 hydrogens on the atom are replaced.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

“Therapeutically effective amount” is intended to include an amount of a compound of the present invention or an amount of the combination of compounds claimed effective to inhibit a desired metalloprotease in a host. The combination of compounds is preferably a synergistic combination. Synergy, as described for example by Chou and Talalay, Adv. Enzyme Regul. 1984, 22, 27-55, occurs when the effect (in this case, inhibition of the desired target) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased anti-inflammatory effect, or some other beneficial effect of the combination compared with the individual components.

Utility

By “administered in combination” or “combination therapy” it is meant that a compound of Formula (I), (II), or (III) of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect. A combination therapy of a TACE inhibitor and a COX-2 inhibitor for the treatment of an arthritic or inflammatory disorder in a mammal can be evaluated as described in the following tests.

Induction and Assessment of collagen Induced Arthritis in Mice

Arthritis is induced in 8-12 week old male DBA/1 mice by injection of 50 mg of chick type II collagen (CII) in complete Freunds adjuvant (Sigma) on day 0 at the base of the tail as previously described [J. Stuart, Annual Rev. Immunol., 1984, 2, 199]. Compounds are prepared as a suspension in 0.5% methylcellulose (Sigma, St. Louis, Mo.), 0.025% Tween 20 (Sigma). The TACE inhibitor and COX-2 inhibitor are administered alone or in combination. The compounds are administered in non-arthritic animals by gavage in a volume of 0.1 ml beginning on day 20 post collagen injection and continuing daily until final evaluation on day 55.

Animals are boosted on day 21 with 50 mg of collagen (CII) in incomplete Freunds adjuvant. The animals are subsequently evaluated several times each week for incidence and severity of arthritis until approximately day 56. Any animal with paw redness or swelling is counted as arthritic. Scoring of severity is carried out using a score of 0-3 for each paw (maximal score of 12/mouse) as previously described [P. Wooley, et al., Trans. Proc., 1983, 15, 180]. The animals are measured for incidence of arthritis and severity in the animals where arthritis is observed. The incidence of arthritis is determined at a gross level by observing the swelling or redness in the paw or digits. Severity is measured with the following guidelines. Briefly, animals displaying four normal paws, i.e., no redness or swelling are scored 0. Any redness or swelling of digits or the paw is scored as 1. Gross swelling of the whole paw or deformity is scored as 2. Ankylosis of joints is scored as 3.

Histological Examination of Paws

In order to verify the gross determination of a non-arthritic animal, a histological examination is performed. Paws from animals sacrificed at the end of the experiment were removed, fixed and decalcified as previously described [R. Jonsson, J. Immunol. Methods, 1986, 88, 109]. Samples are paraffin embedded, sectioned, and stained with hernatoxylin and eosin by standard methods. Stained sections are examined for cellular infiltrates, synovial hyperplasia, and bone and cartilage erosion.

Rat Carrageenan Foot Pad Edema Test

The carrageenan foot edema test is performed with materials, reagents and procedures essentially as described by Winter et al., (Proc. Soc. ExR. Biol. Med., 1962, 111, 544). Male Sprague-Dawley rats are selected in each group so that the average body weight is as close as possible. Rats are fasted with free access to water for over L/ sixteen hours prior to the test. The rats are dosed orally (I m 1L) with compounds suspended in vehicle containing 0.5% methylcellulose and 0.025% surfactant, or with vehicle alone. One hour later a subplantar injection of 0.1 mL of 1% solution of carrageenan/sterile 0.9% saline is administered and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Ottemess and Bliven, Laboratoly Models for Testing NSAIDs, in Non-steroidal Anti-Inflammatory, D. J. Lombardino, ed. 1985)).

Rat Carrageenan-Induced Analgesia Test

The analgesia test using rat carrageenan is performed with materials, reagents and procedures essentially as described by Hargreaves, et al., (Lain, 2, 77 (1988)). Male Sprague-Dawley rats are treated as previously described for the Carrageenan Foot Pad Edema test. Three hours after the injection of the carrageenan, the rats are placed in a special plexiglass container with a transparent floor having a high intensity lamp as a radiant heat source, positionable under the floor. After an initial twenty minute period, thermal stimulation is begun on either the injected foot or on the contralateral uninjected foot. A photoelectric cell turns off the lamp and timer when light is interrupted by paw withdrawal. The time until the rat withdraws its foot is then measured. The withdrawal latency in seconds is detem-lined for the control and drug-treated groups, and percent inhibition of the hyperalgesic foot withdrawal detem-fined.

TNF-α sequestration agents that may be used in combination with a compound of Formula (I), (II), or (III) of this invention, are TNF-α binding proteins or anti-TNF-α antibodies. These agents include, but are not limited to, etanercept (Enbrel), infliximab (Remicade), adalimumab (D2E7), CDP-571 (Humicade), and CDP-870.

Other anti-inflammatory agents that may be used in combination with the compounds of this invention, include, but are not limited to, methotrexate, interleukin-1 antagonists (e.g., anakinra (Kineret)), dihydroorotate synthase inhibitors (e.g., leflunomide (Arava)), and p38 MAP kinase inhibitors.

Although this invention has been described with respect to specific embodiments, the details of these embodiments are not to be construed as limitations.

Dosage and Formulation

The TACE inhibitor (component (i)) and one or more anti-inflammatory agents (component (ii)) combination treatment of this invention can be administered by any conventional means available for the use in conjunction with pharmaceuticals, either as individual separate dosage units administered simultaneously or concurrently, or in a physical combination of each component therapeutic agent in a single or combined dosage unit. The active agents can be administered alone, but are generally administered with a pharmaceutical carrier selected on the basis of the chose route of administration and standard pharmaceutical practice.

The dosage administered will, of course vary depending on their use and known factors such as the pharmacodynamic characteristics of the particular agent, and its mode and route of administration; age, health, and weight of the recipient; nature and extent of symptoms, kind of concurrent treatment, frequency of treatment, and the effect desired. A physician or veterinarian can determine and prescribe the effective amount of the drug required to prevent, counter, or arrest the progress of the thromboembolic disorder.

For therapeutic use, purified soluble tumor necrosis factor receptor antagonist is administered to a patient, preferably a human, for treatment of an inflammation disorder, for example arthritis. Thus, for example, soluble tumor necrosis factor receptor antagonist compositions can be administered by parental administration, for example, intravenous injection, subcutaneous injection, intramuscular injection, or intramedullary injection. Other routes of administration for tumor necrosis factor receptor antagonizing agents include, for example, intra-articular, intraperitoneal or subcutaneous routes by bolus injection, continuous infusion, sustained release from implants, or other suitable techniques. Typically, a soluble tumor necrosis factor receptor therapeutic agent will be administered in the form of a composition comprising purified protein in conjunction with physiologically acceptable carriers, excipients or diluents. Such carriers will be nontoxic to recipients at the dosages and concentrations employed. Ordinarily, the preparation of such compositions entails combining the tumor necrosis factor receptor with buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. Preferably, product is formulated as a lyophilizate using appropriate excipient solutions (e.g., sucrose) as diluents. Appropriate dosages can be determined in trials. In accordance with appropriate industry standards, preservatives may also be added, such as benzyl alcohol. The amount and frequency of administration will depend, of course, on such factors as the nature and severity of the indication being treated, the desired response, the condition of the patient, and so forth.

For treatment of arthritis or an inflammatory disorder, tumor necrosis factor receptor antagonizing agent is administered in systemic amounts ranging from about 0.1 mg/kg/week to about 100 mg/kg/week. In one embodiment of the present invention, tumor necrosis factor receptor antagonizing agent is administered in amounts ranging from about 0.5 mg/kg/week to about 50 mg/kg/week. For local intra-articular administration, dosages preferably range from about 0.01 mg/kg to about 1.0 mg/kg per injection.

Dosage levels of cyclooxygenase-2 inhibitors on the order of about 0.1 mg to about 10,000 mg of the active ingredient compound are useful in the treatment of the above conditions, with preferred levels of about 0.1 mg to about 1,000 mg. The amount of active ingredient that may be combined with other agents to produce a single dosage form will vary depending upon the host treated and the particular mode of administration.

Suitable combination products of this invention can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

Combination products of the present invention are typically administered in admixture with suitable pharmaceutical diluents, excipients, or carriers (collectively referred to herein as pharmaceutical carriers) suitably selected with respect to the intended form of administration, that is, oral tablets, capsules, elixirs, syrups, suspension and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Combination products of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Combination products of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

Each therapeutic agent component of this invention can independently be in any dosage form, such as those described above, and can also be administered in various ways, as described above. The component (i) and (ii) of the invention may be formulated together, in a single dosage unit (that is, combined together in one capsule, tablet, powder, or liquid, etc.) as a combination product. When component (i) and (ii) are not formulated together in a single dosage unit, the TACE inhibitor component (i) may be administered at the same time as the second component (ii) or in any order; for example component (i) of this invention may be administered first, followed by administration of component (ii), or they may be administered in the reverse order. When not administered at the same time, preferably the administration of component (i) and (ii) of this invention occurs less than about one hour apart. Preferably, the route of administration of component (i) and (ii) of the invention is oral. The terms oral agent, oral inhibitor, oral compound, or the like, as used herein, denote compounds which may be orally administered. Although it is preferable that component (i) and component (ii) of the invention are both administered by the same route (that is, for example, both orally) or dosage form, if desired, they may each be administered by different routes (that is, for example, one component of the combination product may be administered orally, and another component may be administered intravenously) or dosage forms. As is appreciated by a medical practitioner skilled in the art, the dosage of the combination therapy of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired, as described above.

The proper dosage of components (i) and (ii) in this invention will be readily ascertainable by a medical practitioner skilled in the art, based upon the present disclosure. By way of general guidance, typically a daily dosage may be about 100 milligrams to about 1.5 grams of each component. By way of general guidance, when the compounds of component (i) and component (ii) are administered in combination, the dosage amount of each component may be reduced by about 70-80% relative to the usual dosage of the component when it is administered alone as a single agent for the treatment of inflammatory diseases, in view of the synergistic effect of the combination.

The combination products of this invention may be formulated such that, although the active ingredients are combined in a single dosage unit, the physical contact between the active ingredients is minimized. In order to minimize contact, for example, where the product is orally administered, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. Another embodiment of this invention where oral administration is desired provides for a combination product wherein one of the active ingredients is coated with a sustained-release material which effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose or other appropriate materials as known in the art, in order to further separate the active components. The polymer coating serves to form an additional barrier to interaction with the other component.

Dosage forms of the combination products of the present invention wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-perils, which are then enteric coated. These enteric coated microtablets, particles, granules or non-perils are then placed into a capsule or compressed into a capsule along with a granulation of the other active ingredient.

These as well as other ways of minimizing contact between the components of combination products of the present invention, whether administered in a single dosage form or administered in separate forms but at the same time or concurrently by the same manner, will be readily apparent to those skilled in the art, based on the present disclosure.

Pharmaceutical kits useful for the treatment of inflammatory diseases, which comprise a therapeutically effective amount of a pharmaceutical composition comprising a compound of component (i) and a compound of component (ii), in one or more sterile containers, are also within the ambit of the present invention. Sterilization of the container may be carried out using conventional sterilization methodology well known to those skilled in the art. Component (i) and component (ii) may be in the same sterile container or in separate sterile containers. The sterile containers of materials may comprise separate containers, or one or more multi-part containers, as desired. Component (i) and component (ii), may be separate, or physically combined into a single dosage form or unit as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

What is claimed is:

1. A method for treating an inflammatory disease in a mammal in need thereof, comprising administering to the mammal a therapeutically effective amount of a combination of: (i) at least one TACE inhibitor, and (ii) one or more anti-inflammatory agents selected from the group consisting of: selective COX-2 inhibitors, interleukin-1 antagonists, dihydroorotate synthase inhibitors, p38 MAP kinase inhibitors, TNF-α inhibitors, TNF-α sequestration agents, and methotrexate; wherein the TACE inhibitor is selected from compounds of Formula (I), (II), (III), or (IV): 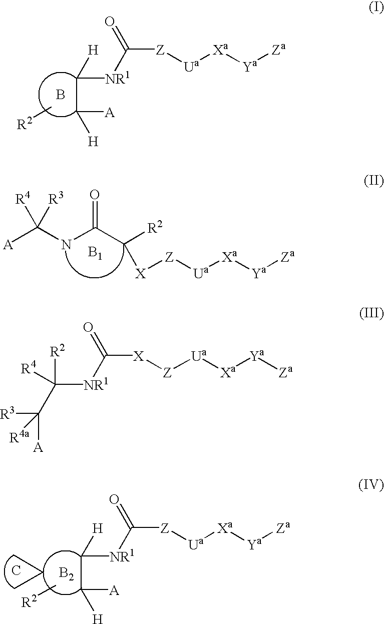
or a stereoisomer or pharmaceutically acceptable salt form thereof, wherein;
A, at each occurrence, is independently selected from —COR⁵, —CO₂H, CH₂CO₂H, —CONHOH, —CONHOR⁵, —CONHOR⁶, —N(OH)COR⁵, —SH, and —CH₂SH;
ring B is a 4-7 membered non-aromatic ring comprising: carbon atoms, 0-1 carbonyl groups, 0-1 double bonds, and from 0-2 ring heteroatoms selected from O, N, and NR² and substituted with 0-3 R^{c}; provided that ring B contains other than a O—O or N—O bond;
ring B₁ is a 4-7 membered cyclic amide containing from 0-2 additional heteroatoms selected from O, NR², and S(O)ₚ, and 0-1 additional carbonyl groups and 0-1 vinyl groups;
ring B₂ is a 4-7 membered non-aromatic carbocyclic or heterocyclic ring comprising: carbon atoms, 0-1 carbonyl groups, 0-1 double bonds, and from 0-2 ring heteroatoms selected from O, N, and NR², provided that ring B contains other than a O—O bond;
ring C forms a spiro ring on Ring B₂ and is a 4-10 membered carbocycle substituted with 0-3 R^{g} or a 4-10 membered heterocycle comprising: carbon atoms, 0-3 carbonyl groups, 0-4 double bonds, and from 0-4 ring heteroatoms selected from O, N, NR², and S(O)ₚ and substituted with 0-3 R^{g}, provided that ring C contains other than a S—S, O—O, or S—O bond;
X, at each occurrence, is absent or is independently selected from C₁₋₄ alkylene, C₂₋₄ alkenylene, C₂₋₄ alkynylene;
Z, at each occurrence, is absent or is independently selected from a C₃₋₆ carbocycle substituted with 0-4 R^{b} and a 5-6 membered heterocyclic system containing from 1-4 heteroatoms selected from the group consisting of N, O, and S and substituted with 0-5 R^{b};
U^{a}, at each occurrence, is absent or is independently selected from: O, NR^{a}, C(O), C(O)O, C(O)NR^{a}, NR^{a}C(O), S(O)ₚ, and S(O)ₚNR^{a};
X^{a}, at each occurrence, is absent or is independently selected from C₁₋₄ alkylene, C₂₋₄ alkenylene, and C₂₋₄ alkynylene;
Y^{a}, at each occurrence, is absent or is independently selected from O and NR^{a};
Z^{a}, at each occurrence, is absent or is independently selected from H, a C₃₋₁₀ carbocycle substituted with 0-5 R^{c} and a 5-10 membered heterocyclic system containing from 1-4 heteroatoms selected from the group comprising N, O, and S(O)ₚ and substituted with 0-5 R^{c}; provided that Z, U^{a}, Y^{a}, and Z^{a} do not combine to form a N—N,N—O, O—N,O—O, S(O)ₚ—O, O—S(O)ₚ or S(O)ₚ—S(O)ₚ group;
R¹, at each occurrence, is independently selected from H, C₁₋₄ alkyl, phenyl and benzyl;
R², at each occurrence, is independently selected from Q, C₁₋₆ alkylene-Q, C₂₋₆ alkenylene-Q, C₂₋₆ alkynylene-Q, (CR^{a}R^{a1})ᵣ₁O(CR^{a}R^{a1})ᵣ-Q, (CR^{a}R^{a1})ᵣ₁NR^{a}(CR^{a}R^{a1})ᵣ-Q, (CR^{a}R^{a1})ᵣ₁C(O)(CR^{a}R^{a1})ᵣ-Q, (CR^{a}R^{a1})ᵣ₁C(O)O(CR^{a}R^{a1})ᵣ-Q, (CR^{a}R^{a1})ᵣC(O)NR^{a}R^{a1}, (CR^{a}R^{a1})ᵣ₁C(O)NR^{a}(CR^{a}R^{a1})ᵣ-Q, (CR^{a}R^{a1})ᵣ₁S(O)ₚ(CR^{a}R^{a1})ᵣ-Q, and (CR^{a}R^{a1})ᵣ₁SO₂NR^{a}(CR^{a}R^{a1})ᵣ-Q;
R³, at each occurrence, is independently selected from H, C₁₋₆ alkylene-Q, C₂₋₆ alkenylene-Q, C₂₋₆ alkynylene-Q, (CH₂)ᵣ₁O(CH₂)ᵣ-Q, (CH₂)ᵣ₁NR^{a}(CH₂)ᵣ-Q, (CH₂)ᵣ₁C(O)(CH₂)ᵣ-Q, (CH₂)ᵣ₁C(O)NR^{a}(CH₂)ᵣ-Q, (CH₂)ᵣ₁NR^{a}C(O)(CH₂)ᵣ-Q, (CH₂)ᵣ₁OC(O)NR^{a}(CH₂)ᵣ-Q, (CH₂)ᵣ₁NR^{a}C(O)O(CH₂)ᵣ-Q, (CH₂)ᵣ₁NR^{a}C(O)NR^{a}(CH₂)ᵣ-Q, (CH₂)ᵣ₁S(O)ₚ(CH₂)ᵣ-Q, (CH₂)ᵣ₁SO₂NR^{a}(CH₂)ᵣ-Q, (CH₂)ᵣ₁NR^{a}SO₂(CH₂)ᵣ-Q, and (CH₂)ᵣ₁NR^{a}SO₂NR^{a}(CH₂)ᵣ-Q;
Q, at each occurrence, is independently selected from H, a C₃₋₆ carbocycle substituted with 0-5 R^{d} and a 5-10 membered heterocyclic system containing from 1-4 heteroatoms selected from the group consisting of N, O, and S and substituted with 0-5 R^{d};
R⁴, at each occurrence, is independently selected from H and C₁₋₆ alkyl;
R^{4a} is selected from H and C₁₋₆ alkyl;
alternatively, R³ and R⁴ in Formula (II) together with the carbon atom to which they are attached combine to form a 3-6 membered carbocyclic or heterocyclic ring comprising carbon atoms and 0-2 ring heteroatoms selected from O, N, NR^{c}, and S(O)ₚ and substituted with 0-1 R^{c};
alternatively, R¹ and R² in Formula (III) together with the carbon and nitrogen atoms to which they are attached combine to form a 3-10 membered heterocyclic ring comprising carbon atoms and, in addition to the nitrogen atom to which R¹ is attached, 0-1 ring heteroatoms selected from O, N, NR^{c}, and S(O)ₚ and substituted with 0-1 R^{c};
alternatively, R¹ and R³ in Formula (III) together with the carbon and nitrogen atoms to which they are attached combine to form a 4-6 membered heterocyclic ring comprising carbon atoms and, in addition to the nitrogen atom to which R¹ is attached, 0-1 ring heteroatoms selected from O, N, and NR^{c}, and substituted with 0-1 R^{c};
alternatively, R² and R⁴ in Formula (III) together with the carbon atom to which they are attached combine to form a 3-10 membered carbocyclic or heterocyclic ring comprising carbon atoms and 0-2 ring heteroatoms selected from O, N, NR^{c}, and S(O)ₚ and substituted with 0-3 R^{c};
alternatively, R³ and R^{4a} in Formula (III) together with the carbon atom to which they are attached combine to form a 3-6 membered carbocyclic or heterocyclic ring comprising carbon atoms and 0-2 ring heteroatoms selected from O, N, NR^{c}, and S(O)ₚ and substituted with 0-1 R^{c};
R⁵, at each occurrence, is selected from C₁₋₆ alkyl substituted with 0-2 R^{b}, and C₁₋₄ alkyl substituted with 0-2 R^{e};
R⁶, at each occurrence, is selected from phenyl, naphthyl, C₁₋₁₀ alkyl-phenyl-C₁₋₆ alkyl-, C₃₋₁₁ cycloalkyl, C₁₋₆ alkylcarbonyloxy-C₁₋₃ alkyl-, C₁₋₆ alkoxycarbonyloxy-C₁₋₃ alkyl-, C₂₋₁₀ alkoxycarbonyl, C₃₋₆ cycloalkylcarbonyloxy-C₁₋₃ alkyl-, C₃₋₆ cycloalkoxycarbonyloxy-C₁₋₃ alkyl-, C₃₋₆ cycloalkoxycarbonyl, phenoxycarbonyl, phenyloxycarbonyloxy-C₁₋₃ alkyl-, phenylcarbonyloxy-C₁₋₃ alkyl-, C₁₋₆ alkoxy-C₁₋₆ alkylcarbonyloxy-C₁₋₃ alkyl-, [5-(C₁-C₅ alkyl)-1,3-dioxa-cyclopenten-2-one-yl]methyl, [5-(R^{a})-1,3-dioxa-cyclopenten-2-one-yl]methyl, (5-aryl-1,3-dioxa-cyclopenten-2-one-yl)methyl, —C₁₋₁₀ alkyl-NR⁷R^{7a}, —CH(R⁸)OC(═O)R⁹, and —CH(R⁸)OC(═O)OR⁹;
R⁷, at each occurrence, is independently selected from H and C₁₋₆ alkyl, C₂₋₆ alkenyl, C₃₋₆ cycloalkyl-C₁₋₃ alkyl-, and phenyl-C₁₋₆ alkyl-;
R^{7a}, at each occurrence, is independently selected from H and C₁₋₆ alkyl, C₂₋₆ alkenyl, C₃₋₆ cycloalkyl-C₁₋₃ alkyl-, and phenyl-C₁₋₆ alkyl-;
R⁸, at each occurrence, is independently selected from H and C₁₋₄ linear alkyl;
R⁹, at each occurrence, is independently selected from H, C₁₋₆ alkyl substituted with 1-2 R^{f}, C₃₋₆ cycloalkyl substituted with 1-2 R^{f}, and phenyl substituted with 0-2 R^{b};
R^{a}, at each occurrence, is independently selected from H, C₁₋₄ alkyl, phenyl and benzyl;
R^{a1}, at each occurrence, is independently selected from H and C₁₋₄ alkyl;
alternatively, R^{a} and R^{a1} when attached to a nitrogen are taken together with the nitrogen to which they are attached to form a 5 or 6 membered ring comprising carbon atoms and from 0-1 additional heteroatoms selected from the group consisting of N, O, and S; R^{a2}, at each occurrence, is independently selected from C₁₋₄ alkyl, phenyl, and benzyl;
R^{b}, at each occurrence, is independently selected from C₁₋₆ alkyl, OR^{a}, Cl, F, Br, ═O, —CN, NR^{a}R^{a1}, C(O)R^{a}, C(O)OR^{a}, C(O)NR^{a}R^{a1}, S(O)₂NR^{a}R^{a1}, S(O)ₚR^{a1}, and CF₃;
R^{c}, at each occurrence, is independently selected from C₁₋₆ alkyl, OR^{a}, Cl, F, Br, ═O, —CN, NR^{a}R^{a1}, C(O)R^{a}, C(O)OR^{a}, C(O)NR^{a}R^{a1}, S(O)₂NR^{a}R^{a1}, S(O)ₚR^{a1}, CF₃, (CH₂)ᵣ—C₃₋₆ carbocycle and a (CH₂)ᵣ-5-6 membered heterocycle comprising: carbon atoms and 1-4 heteroatoms selected from the group consisting of N, O, and S;
alternatively, two R^{c}s on the same carbon atom are taken together with the carbon atom to which they are attached to form a 5 or 6 membered ring comprising carbon atoms and from 0-1 additional heteroatoms selected from the group consisting of N, O, and S;
R^{c1}, at each occurrence, is independently selected from C₁₋₆ alkyl, OR^{a}, Cl, F, Br, I, ═O, —CN, NO₂, NR^{a}R^{a1}, C(O)R^{a}, C(O)OR^{a}, C(O)NR^{a}R^{a1}, R^{a}NC(O)NR^{a}R^{a1}, OC(O)NR^{a}R^{a1}, R^{a}NC(O)OR^{a}, S(O)₂NR^{a}R^{a1}, NR^{a}S(O)₂R^{a2}, NR^{a}S(O)₂NR^{a}R^{a1}, OS(O)₂NR^{a}R^{a1}, NR^{a}S(O)₂R^{a2}, S(O)ₚR^{a2}, CF₃, CF₂CF₃, CH₂F, and CHF₂;
R^{d}, at each occurrence, is independently selected from C₁₋₆ alkyl, OR^{a}, Cl, F, Br, ═O, —CN, NR^{a}R^{a1}, C(O)R^{a}, C(O)OR^{a}, C(O)NR^{a}R^{a1}, S(O)₂NR^{a}R^{a1}, S(O)ₚR^{a2}, CF₃, C₃₋₆ carbocyclic residue and a 5-6 membered heterocycle comprising: carbon atoms and 1-4 heteroatoms selected from the group consisting of N, O, and S;
R^{e}, at each occurrence, is selected from phenyl substituted with 0-2 R^{b} and biphenyl substituted with 0-2 R^{b};
R^{f}, at each occurrence, is selected from C₁₋₄ alkyl, C₃₋₆ cycloalkyl, C₁₋₅ alkoxy, phenyl substituted with 0-2 R^{b};
R^{g}, at each occurrence, is independently selected from C₁₋₆ alkyl, OR^{a}, Cl, F, Br, I, ═O, —CN, NO₂, NR^{a}R^{a1}, C(O)R^{a}, C(O)OR^{a}, C(O)NR^{a}R^{a1}, R^{a}NC(O)NR^{a}R^{a1}, OC(O)NR^{a}R^{a1}, R^{a}NC(O)OR^{a}, S(O)₂NR^{a}R^{a1}, NR^{a}S(O)₂R^{a2}, NR^{a}S(O)₂NR^{a}R^{a1}, OS(O)₂NR^{a}R^{a1}, NR^{a}S(O)₂R^{a2}, S(O)ₚR^{a2}, CF₃, CF₂CF₃, C₃₋₁₀ carbocycle substituted with 0-2 R^{c1}, (CR^{a}R^{a1})ᵣ₁—C₃₋₁₀ carbocycle substituted with 0-2 R^{c1}, a 5-14 membered heterocycle comprising carbon atoms and 1-4 heteroatoms selected from the group consisting of N, O, and S(O)ₚ and substituted with 0-2 R^{c1}, and (CR^{a}R^{a1})ᵣ₁-5-14 membered heterocycle comprising carbon atoms and 1-4 heteroatoms selected from the group consisting of N, O, and S(O)ₚ and substituted with 0-2 R_{c1};
p, at each occurrence, is selected from 0, 1, and 2;
r, at each occurrence, is selected from 0, 1, 2, 3, and 4; and
r1, at each occurrence, is selected from 0, 1, 2, 3, and 4.

2. The method of claim 1 wherein the TACE inhibitor is a compound selected from:
[1(R)]-3-amino-N-hydroxy-α-(2-methylpropyl)-2-oxo-3-[4-(2-quinolinylmethoxy)phenyl]-1-pyrrolidineacetamide;
[1(R)]-3-amino-N-hydroxy-α-(2-methylpropyl)-2-oxo-3-[4-(4-quinolinylmethoxy)phenyl]-1-pyrrolidineacetamide;
[1(R)]-3-amino-N-hydroxy-3-[4-[(2-methoxy-4-quinolinyl)methoxy]phenyl]-α-(2-methylpropyl)-2-oxo-1-pyrrolidineacetamide;
[1(R)]-3-amino-N-hydroxy-α-(2-methylpropyl)-2-oxo-3-[4-[(2-phenyl-4-quinolinyl)methoxy]phenyl]-1-pyrrolidineacetamide;
[1(R)]-3-amino-3-[4-[(2,6-dimethyl-4-quinolinyl)methoxy]phenyl]-N-hydroxy-α-(2-methylpropyl)-2-oxo-1-pyrrolidineacetamide;
[1(R)]-3-amino-3-[4-[(2-chloro-4-quinolinyl)methoxy]phenyl]-N-hydroxy-α-(2-methylpropyl)-2-oxo-1-pyrrolidineacetamide;
[1(R)]-3-amino-N-hydroxy-a-(2-methylpropyl)-3-[4-[(2-methyl-4-quinolinyl)methoxy]phenyl]-2-oxo-1-pyrrolidineacetamide;
[1(R)]-3-amino-3-[4-[(2-chloro-4-quinolinyl)methoxy]phenyl]-N-hydroxy-α-[2-(methylsulfonyl)ethyl]-2-oxo-1-pyrrolidineacetamide;
[1(R)]-3-amino-N-hydroxy-3-[4-[(2-methyl-4-quinolinyl)methoxy]phenyl]-α-[2-(methylsulfonyl)ethyl]-2-oxo-1-pyrrolidineacetamide;
[1(R)]-3-amino-N-hydroxy-3-[4-[(2-methoxy-4-quinolinyl)methoxy]phenyl]-α-[2-(methylsulfonyl)ethyl]-2-oxo-1-pyrrolidineacetamide;
[1(R)]-N-hydroxy-3-(methylamino)-α-(2-methylpropyl)-3-[4-[(2-methyl-4-quinolinyl)methoxy]phenyl]-2-oxo-1-pyrrolidineacetamide;
[1(R)]-α-[3-amino-2-oxo-3-[4-(4-quinolinylmethoxy)phenyl]-1-pyrrolidinyl]-N-hydroxy-4-piperidineacetamide;
[1(R)]-3-amino-N-hydroxy-α-(1-methylethyl)-2-oxo-3-[4-(4-quinolinylmethoxy)phenyl]-1-pyrrolidineacetamide;
[1(R)]-3-amino-α-cyclohexyl-N-hydroxy-2-oxo-3-[4-(4-quinolinylmethoxy)phenyl]-1-pyrrolidineacetamide;
[1(R)]-3-amino-α-(1,1-dimethylethyl)-N-hydroxy-2-oxo-3-[4-(4-quinolinylmethoxy)phenyl]-1-pyrrolidineacetamide;
[1(R)]-3-amino-α-(1,1-dimethylethyl)-N-hydroxy-2-oxo-3-[4-[(2-methyl-4-quinolinyl)methoxy]phenyl]-1-pyrrolidineacetamide;
[1(R)]-3-amino-N-hydroxy-α-(1-methylethyl)-2-oxo-3-[4-[(2-methyl-4-quinolinyl)methoxy]phenyl]-1-pyrrolidineacetamide;
[1(R)]-3-amino-N-hydroxy-α-(1-methylethyl)-2-oxo-3-[4-[(2,6-dimethyl-4-quinolinyl)methoxy]phenyl]-1-pyrrolidineacetamide;
(3S,4S)-N-hydroxy-1-isopropyl-4-({4-[(2-methyl-4-quinolinyl)methoxy]benzoyl}amino)-3-pyrrolidinecarboxamide;
(3S,4S)-N-hydroxy-4-({4-[(2-methyl-4-quinolinyl)methoxy]benzoyl}amino)-1-(2-propynyl)-3-pyrrolidinecarboxamide;
(3S,4S)-1-(2-butynyl)-N-hydroxy-4-({4-[(2-methyl-4-quinolinyl)methoxy]benzoyl}amino)-3-pyrrolidinecarboxamide;
(3R,4S)-N-hydroxy-4-({4-[(2-methyl-4-quinolinyl)methoxy]benzoyl}amino)tetrahydro-3-furancarboxamide;
(3S,4S)-4-{[4-(2-butynyloxy)benzoyl]amino}-N-hydroxy-1-isopropyl-3-pyrrolidinecarboxamide;
(1S,2R)-N-hydroxy-2-[[[4-[(2-methyl-4-quinolinyl)methoxy]phenyl]carbonyl]amino]-1-cyclopentanecarboxamide;
(3S,4R)-N-hydroxy-1-methyl-4-[[[4-[(2-methyl-4-quinolinyl)methoxy]phenyl]carbonyl]amino]-3-piperidinecarboxamide;
(3S,4S)-N-hydroxy-1-(1-methylethyl)-3-[[[4-[(2-methyl-4-quinolinyl)methoxy]phenyl]carbonyl]amino]-4-piperidinecarboxamide;
(3R,4R)-N-hydroxy-4-({4-[(2-methyl-4-quinolinyl)methoxy]benzoyl}amino)tetrahydro-2H-pyran-3-carboxamide;
(3S,4S)-1-tert-butyl-N-hydroxy-3-({4-[(2-methyl-4-quinolinyl)methoxy]benzoyl}amino)-4-piperidinecarboxamide;
(3S,4S)-3-({4-[(2,5-dimethylbenzyl)oxy]benzoyl}amino)-N-hydroxy-4-piperidinecarboxamide;
N-{4-[2-(hydroxyamino)-2-oxoethyl]-4-piperidinyl}-4-[(2-methyl-4-quinolinyl)methoxy]benzamide,
N-[3-[2-(hydroxyamino)-2-oxoethyl]-1-(4-pyridinylmethyl)-3-piperidinyl]-4-[(2-methyl-4-quinolinyl)methoxy]benzamide,
N-{4α-[2-(hydroxyamino)-2-oxoethyl]-2β,6β-dimethyl-4-piperidinyl}-4-[(2-methyl-4-quinolinyl)methoxy]benzamide,
N-{4-[2-(hydroxyamino)-2-oxoethyl]hexahydro-1H-azepin-4-yl}-4-[(2-methyl-4-quinolinyl)methoxy]benzamide,
N-{4-[2-(hydroxyamino)-2-oxoethyl]tetrahydro-2H-pyran-4-yl}-4-[(2-methyl-4-quinolinyl)methoxy]benzamide,
N-{(3R)-3-[2-(hydroxyamino)-2-oxoethyl]tetrahydro-2H-pyran-3-yl}-4-[(2-methyl-4-quinolinyl)methoxy]benzamide,
N-{(3S)-3-[2-(hydroxyamino)-2-oxoethyl]tetrahydro-2H-pyran-3-yl}-4-[(2-methyl-4-quinolinyl)methoxy]benzamide,
N-{4-[2-(hydroxyamino)-2-oxoethyl] tetrahydro-2H-thiopyran-4-yl}-4-[(2-methyl-4-quinolinyl)methoxy]benzamide,
N-{4-[2-(hydroxyamino)-2-oxoethyl]-1,1-dioxidotetrahydro-2H-thiopyran-4-yl}-4-[(2-methyl-4-quinolinyl)methoxy]benzamide,
N-{3-[2-(hydroxyamino)-2-oxoethyl]tetrahydro-3-furanyl}-4-[(2-methyl-4-quinolinyl)methoxy]benzamide,
(5R,7S,8R)-N-hydroxy-8-({4-[(2-methyl-4-quinolinyl)methoxy]benzoyl}amino)-1-oxaspiro[4.4]nonane-7-carboxamide,
(5S,7S,8R)-N-hydroxy-8-({4-[(2-methyl-4-quinolinyl)methoxy]benzoyl}amino)-1-oxaspiro[4.4]nonane-7-carboxamide,
(5R,7S,8R)-8-{[4-(2-butynyloxy)benzoyl]amino}-N-hydroxy-1-oxaspiro[4.4]nonane-7-carboxamide,
(5R,7S,8R)-N-hydroxy-8-({4-[(2-isopropyl-1H-benzimidazol-1-yl)methyl]benzoyl}amino)-1-oxaspiro[4.4]nonane-7-carboxamide,
(5R,7S,8R)-N-hydroxy-8-[(4-{[2-(trifluoromethyl)-1H-benzimidazol-1-yl]methyl}benzoyl)amino]-1-oxaspiro[4.4]nonane-7-carboxamide,
(5R,7S,8R)-8-[(4-{[2-(1,1-difluoroethyl)-1H-benzimidazol-1-yl]methyl}benzoyl)amino]-N-hydroxy-1-oxaspiro[4.4]nonane-7-carboxamide,
(5R,7S,8R)-8-({4-[(3,5-dimethyl-1H-pyrazol-4-yl)methyl]benzoyl}amino)-N-hydroxy-1-oxaspiro[4.4]nonane-7-carboxamide,
(5R,7S,8R)-N-hydroxy-8-({4-[(2-methyl-4-quinolinyl)methyl]benzoyl}amino)-1-oxaspiro[4.4]nonane-7-carboxamide,
(5R,7S,8R)-N-hydroxy-8-[(4-{[2-(trifluoromethyl)-4-quinolinyl]methyl}benzoyl)amino]-1-oxaspiro[4.4]nonane-7-carboxamide, and
(5R,7S,8R)-N-hydroxy-8-({4-[(2-isopropyl-4-quinolinyl)methyl]benzoyl}amino)-1-oxaspiro[4.4]nonane-7-carboxamide,
or a pharmaceutically acceptable salt form thereof.

3. The method of claim 1 wherein at least one of the anti-inflammatory agents of the component (ii) is selective COX-2 inhibiting agent.

4. The method of claim 3 wherein the selective COX-2 inhibiting agent is selected from:
4-[4-(methyl-sulfonyl)phenyl]-3-phenyl-2(5H)-furanone (rofecoxib);
4-(5-(4-methylphenyl)-3-trifluoromethyl)-1H-pyrazol-1-yl)-benzenesulfonamide (celecoxib);
6-chloro-4-hydroxy-2-methyl-N-2-pyridinyl-2H-thieno[2,3-e]-1,2-thiazine-3-carboxamide-1,1-dioxide (lornoxicam);
4-hydroxy-2-methyl-N-(5-methyl-2-thiazolyl)-1,1-dioxide-2H-1,2-benzothiazine-3-carboxamide (meloxicam);
N-(4-nitro-2-phenoxyphenyl)methanesulfonamide (nimesulide);
4-(5-methyl-3-phenyl-4-isoxazolyl)-benzenesulfonamide (valdecoxib);
2-(6-methylpyrid-3-yl)-3-(4-methylsulfinylphenyl)-5-chloropyridine (etoricoxib);
N-[[4-(5-methyl-3-phenyl-4-isoxazolyl)phenyl]sulfonyl]-propanamide (parecoxib);
5,5-dimethyl-3-(1-methylethoxy)-4-[4-(methylsulfonyl)phenyl]-2(5H)-furanone (DFP);
N-[6-(2,4-difluorophenoxy)-2,3-dihydro-1-oxo-1H-inden-5-yl] methanesulfonamide (flosulide);
5-methyl-N-[4-(trifluoromethyl)phenyl]-4-isoxazolecarboxamide (leflunomide);
2-fluoro-α-methyl-4-(nitrooxy)butyl ester-[1,1′-biphenyl]-4-acetic acid (nitroflurbiprofen);
1-(4-chlorobenzoyl)-3-[4-(4-fluorophenyl)thiazol-2-ylmethyl]-5-methoxy-2-methylindole (A-183827.0);
4-(4-cyclohexyl-2-methyloxazol-5-yl)-2-fluorobenzenesulfonamide (JTE-522);
3-(3-fluorophenyl)-5,5-dimethyl-4-[4-(methylsulfonyl)phenyl]-2(5H)-furanone (DFU);
3-(3,4-difluorophenoxy)-5-methyl-4-[4-(methylsulfonyl)phenyl]-5-(2,2,2-trifluoroethyl-2(5H)-Furanone (L-784512);
5,5-dimethyl-4-[4-(methylsulfonyl)phenyl]-3-phenoxy-2(5H)-furanone (L-783003);
3-[(5-bromo-2-pyridinyl)oxy]-5,5-dimethyl-4-[4-(methylsulfonyl)phenyl]-2(5H)-furanone (L-778736);
3-[4-(methylsulfonyl)phenyl]-2-(3,5-difluorophenyl)-2-cyclopenten-1-one (L-776967);
5-[4-(methylsulfonyl)-phenyl]-6-phenyl-thiazolo [3,2-b] [1,2,4]triazole (L-768277);
(S)-2-methyl-1-[(4-bromophenyl)methyl]-5-methoxy-β-1H-Indole-3-butanoic acid (L-761066);
N-[6-[(4-ethyl-2-thiazolyl)thio]-1,3-dihydro-1-oxo-5-isobenzofuranyl]methanesulfonamide (L-758115);
5-methoxy-2-methyl-1-(2,4,6-trichlorobenzoyl)-1H-indole-3-acetic acid (L-748780);
4-[2-(4-fluorophenyl)-3-thienyl]-benzenesulfonamide (L-746483);
N-[6-[(2,4-difluorophenyl)thio]-2,3-dihydro-1-oxo-1H-inden-5-yl]-methanesulfonamide (L-745337);
5-[[3,5-bis(1,1-dimethylethyl)-4-hydroxyphenyl]methylene]-2,4-thiazolidinedione (2-(O-methyloxime), PD-138387);
5-(4-fluorophenyl)-6-[4-(methylsulfonyl)phenyl]-spiro[2.4]hept-5-ene (SC-58451);
5-(4-fluorophenyl)-1-[4-(methylsulfonyl)phenyl]-3-(trifluoromethyl)-1H-pyrazole (SC-58231);
1-fluoro-4-[2-[4-(methylsulfonyl)phenyl]cyclopenten-1-yl] benzene (SC-57666);
4-[5-(4-chlorophenyl)-3-(difluoromethyl)-]H-pyrazol-1-yl]benzenesulfonaniide (SC-236);
N-[5-(4-fluoro)phenoxylthlophene-2-methanesulfonamide (RWJ-63556);
6-[[5-(4-chlorobenzoyl)-1,4-dimethyl-1H-pyrrol-2-yl]methyl]-3(2H)-pyridazinone (RS-57067-000);
5(E)-(3,5-di-tert-butyl-4-hydroxy)benzylidene-2-ethyl-1,2-isothiazolidine-1,1-dioxide (S-2474);
3-formylamino-7-methylsulfonylamino-6-phenoxy-4H-1-benzopyran-4-one (T-614);
N-[2-(cyclohexyloxy)-4-nitrophenyl]methanesulfonamide (NS-398);
AF-3161; AF-3162; COX-189; CS 502; CS-179; CT-3; GR-253035; HN-56249; LAS-33815; LAS-33826; LAS-34475; PD-164387; PD-138768; PD-098120; SC-906; SC-58236; S-2474; S-33516; SVT-2016, TP-72; UR-8813; UR-8877; UP-454-21; and UR-8880.

5. The method of claim 1 wherein the inflammatory disease is selected from the group consisting of acute infection, acute phase response, age related macular degeneration, alcoholic liver disease, allergy, allergic asthma, anorexia, aneurism, aortic aneurism, asthma, atherosclerosis, atopic dermatitis, autoimmune disease, autoimmune hepatitis, Bechet's disease, cachexia, calcium pyrophosphate dihydrate deposition disease, cardiovascular effects, chronic fatigue syndrome, chronic obstruction pulmonary disease, coagulation, congestive heart failure, corneal ulceration, Crohn's disease, enteropathic arthropathy, Felty's syndrome, fever, fibromyalgia syndrome, fibrotic disease, gingivitis, glucocorticoid withdrawal syndrome, gout, graft versus host disease, hemorrhage, HIV infection, hyperoxic alveolar injury, infectious arthritis, inflammation, intermittent hydrarthrosis, Lyme disease, meningitis, multiple sclerosis, myasthenia gravis, mycobacterial infection, neovascular glaucoma, osteoarthritis, pelvic inflammatory disease, periodontitis, polymyositis/dermatomyositis, post-ischaemic reperfusion injury, post-radiation asthenia, psoriasis, psoriatic arthritis, pulmonary emphysema, pydoderma gangrenosum, relapsing polychondritis, Reiter's syndrome, rheumatic fever, rheumatoid arthritis, sarcoidosis, scleroderma, sepsis syndrome, Still's disease, shock, Sjogren's syndrome, skin inflammatory diseases, solid tumor growth and tumor invasion by secondary metastases, spondylitis, stroke, systemic lupus erythematosus, ulcerative colitis, uveitis, vasculitis, and Wegener's granulomatosis.

6. A pharmaceutical composition useful for the treatment of an inflammatory disease in a mammal in need thereof, comprising a pharmaceutically acceptable carrier and a therapeutically effective amount of a combination of claim 1.

7. A pharmaceutical kit useful for the treatment of an inflammatory disease in a mammal in need thereof, comprising administering to the mammal a therapeutically effective amount of a combination of claim 1.

8. A method of treating a disease or condition comprising: administering to the mammal in need of such treatment a therapeutically effective amount of a compound of Formula (II): 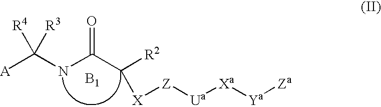
or a stereoisomer or pharmaceutically acceptable salt form thereof, wherein;
A, at each occurrence, is independently selected from —COR⁵, —CO₂H, CH₂CO₂H, —CONHOH, —CONHOR⁵, —CONHOR⁶, —N(OH)COR⁵, —SH, and —CH₂SH;
ring B₁ is a 4-7 membered cyclic amide containing from 0-2 additional heteroatoms selected from O, NR², and S(O)ₚ, and 0-1 additional carbonyl groups and 0-1 vinyl groups;
X, at each occurrence, is absent or is independently selected from C₁₋₄ alkylene, C₂₋₄ alkenylene, C₂₋₄ alkynylene;
Z, at each occurrence, is absent or is independently selected from a C₃₋₆ carbocycle substituted with 0-4 R^{b} and a 5-6 membered heterocyclic system containing from 1-4 heteroatoms selected from the group consisting of N, O, and S and substituted with 0-5 R^{b};
U^{a}, at each occurrence, is absent or is independently selected from: O, NR^{a}, C(O), C(O)O, C(O)NR^{a}, NR^{a}C(O), S(O)ₚ, and S(O)ₚNR^{a};
X^{a}, at each occurrence, is absent or is independently selected from C₁₋₄ alkylene, C₂₋₄ alkenylene, and C₂₋₄ alkynylene;
Y^{a}, at each occurrence, is absent or is independently selected from O and NR^{a};
Z^{a}, at each occurrence, is absent or is independently selected from H, a C₃₋₁₀ carbocycle substituted with 0-5 R^{c} and a 5-10 membered heterocyclic system containing from 1-4 heteroatoms selected from the group comprising N, O, and S(O)ₚ and substituted with 0-5 R^{c};
provided that Z, U^{a}, Y^{a}, and Z^{a} do not combine to form a N—N, N—O, O—N,O—O, S(O)ₚ—O, O—S(O)ₚ or S(O)ₚ—S(O)ₚ group;
R², at each occurrence, is independently selected from Q, C₁₋₆ alkylene-Q, C₂₋₆ alkenylene-Q, C₂₋₆ alkynylene-Q, (CR^{a}R^{a1})ᵣ₁O(CR^{a}R^{a1})ᵣ-Q, (CR^{a}R^{a1})ᵣ₁NR^{a}(CR^{a}R^{a1})ᵣ-Q, (CR^{a}R^{a1})ᵣ₁C(O)(CR^{a}R^{a1})ᵣ-Q, (CR^{a}R^{a1})ᵣ₁C(O)O(CR^{a}R^{a1})ᵣ-Q, (CR^{a}R^{a1})ᵣC(O)NR^{a}R^{a1}, (CR^{a}R^{a1})ᵣ₁C(O)NR^{a}(CR^{a}R^{a1})ᵣ-Q, (CR^{a}R^{a1})ᵣ₁S(O)ₚ(CR^{a}R^{a1})ᵣ-Q, and (CR^{a}R^{a1})ᵣ₁SO₂NR^{a}(CR^{a}R^{a1})ᵣ-Q;
R³, at each occurrence, is independently selected from H, C₁₋₆ alkylene-Q, C₂₋₆ alkenylene-Q, C₂₋₆ alkynylene-Q, (CH₂)ᵣ₁O(CH₂)ᵣ-Q, (CH₂)ᵣ₁NR^{a}(CH₂)ᵣ-Q, (CH₂)ᵣ₁C(O)(CH₂)ᵣ-Q, (CH₂)ᵣ₁C(O)NR^{a}(CH₂)ᵣ-Q, (CH₂)ᵣ₁NR^{a}C(O)(CH₂)ᵣ-Q, (CH₂)ᵣ₁OC(O)NR^{a}(CH₂)ᵣ-Q, (CH₂)ᵣ₁NR^{a}C(O)O(CH₂)ᵣ-Q, (CH₂)ᵣ₁NR^{a}C(O)NR^{a}(CH₂)ᵣ-Q, (CH₂)ᵣ₁S(O)ₚ(CH₂)ᵣ-Q, (CH₂)ᵣ₁SO₂NR^{a}(CH₂)ᵣ-Q, (CH₂)ᵣ₁NR^{a}SO₂(CH₂)ᵣ-Q, and (CH₂)ᵣ₁NR^{a}SO₂NR^{a}(CH₂)ᵣ-Q;
Q, at each occurrence, is independently selected from H, a C₃₋₆ carbocycle substituted with 0-5 R^{d} and a 5-10 membered heterocyclic system containing from 1-4 heteroatoms selected from the group consisting of N, O, and S and substituted with 0-5 R^{d};
R⁴, at each occurrence, is independently selected from H and C₁₋₆ alkyl;
alternatively, R³ and R⁴ together with the carbon atom to which they are attached combine to form a 3-6 membered carbocyclic or heterocyclic ring comprising carbon atoms and 0-2 ring heteroatoms selected from O, N, NR^{c}, and S(O)ₚ and substituted with 0-1 R^{c};
R⁵, at each occurrence, is selected from C₁₋₆ alkyl substituted with 0-2 R^{b}, and C₁₋₄ alkyl substituted with 0-2 R^{e};
R⁶, at each occurrence, is selected from phenyl, naphthyl, C₁₋₁₀ alkyl-phenyl-C₁₋₆ alkyl-, C₃₋₁₁ cycloalkyl, C₁₋₆ alkylcarbonyloxy-C₁₋₃ alkyl-, C₁₋₆ alkoxycarbonyloxy-C₁₋₃ alkyl-, C₂₋₁₀ alkoxycarbonyl, C₃₋₆ cycloalkylcarbonyloxy-C₁₋₃ alkyl-, C₃₋₆ cycloalkoxycarbonyloxy-C₁₋₃ alkyl-, C₃₋₆ cycloalkoxycarbonyl, phenoxycarbonyl, phenyloxycarbonyloxy-C₁₋₃ alkyl-, phenylcarbonyloxy-C₁₋₃ alkyl-, C₁₋₆ alkoxy-C₁₋₆ alkylcarbonyloxy-C₁₋₃ alkyl-, [5-(C₁-C₅ alkyl)-1,3-dioxa-cyclopenten-2-one-yl]methyl, [5-(R^{a})-1,3-dioxa-cyclopenten-2-one-yl]methyl, (5-aryl-1,3-dioxa-cyclopenten-2-one-yl)methyl, —C₁₋₁₀ alkyl-NR⁷R^{7a}, —CH(R⁸)OC(═O)R⁹, and —CH(R⁸)OC(═O)OR⁹;
R⁷, at each occurrence, is independently selected from H and C₁₋₆ alkyl, C₂₋₆ alkenyl, C₃₋₆ cycloalkyl-C₁₋₁₃ alkyl-, and phenyl-C₁₋₆ alkyl-;
R^{7a}, at each occurrence, is independently selected from H and C₁₋₆ alkyl, C₂₋₆ alkenyl, C₃₋₆ cycloalkyl-C₁₋₃ alkyl-, and phenyl-C₁₋₆ alkyl-;
R⁸, at each occurrence, is independently selected from H and C₁₋₄ linear alkyl;
R⁹, at each occurrence, is independently selected from H, C₁₋₆ alkyl substituted with 1-2 R^{f}, C₃₋₆ cycloalkyl substituted with 1-2 R^{f}, and phenyl substituted with 0-2 R^{b};
R^{a}, at each occurrence, is independently selected from H, C₁₋₄ alkyl, phenyl and benzyl;
R^{a1}, at each occurrence, is independently selected from H and C₁₋₄ alkyl;
alternatively, R^{a} and R^{a1} when attached to a nitrogen are taken together with the nitrogen to which they are attached to form a 5 or 6 membered ring comprising carbon atoms and from 0-1 additional heteroatoms selected from the group consisting of N, O, and S;
R^{a2}, at each occurrence, is independently selected from C₁₋₄ alkyl, phenyl, and benzyl;
R^{b}, at each occurrence, is independently selected from C₁₋₆ alkyl, OR^{a}, Cl, F, Br, ═O, —CN, NR^{a}R^{a1}, C(O)R^{a}, C(O)OR^{a}, C(O)NR^{a}R^{a1}, S(O)₂NR^{a}R^{a1}, S(O)ₚR^{a1}, and CF₃;
R^{c}, at each occurrence, is independently selected from C₁₋₆ alkyl, OR^{a}, Cl, F, Br, ═O, —CN, NR^{a}R^{a1}, C(O)R^{a}, C(O)OR^{a}, C(O)NR^{a}R^{a1}, S(O)₂NR^{a}R^{a1}, S(O)ₚR^{a1}, CF₃, (CH₂)ᵣ—C₃₋₆ carbocycle and a (CH₂)ᵣ-5-6 membered heterocycle comprising: carbon atoms and 1-4 heteroatoms selected from the group consisting of N, O, and S;
alternatively, two R^{c}s on the same carbon atom are taken together with the carbon atom to which they are attached to form a 5 or 6 membered ring comprising carbon atoms and from 0-1 additional heteroatoms selected from the group consisting of N, O, and S;
R^{c1}, at each occurrence, is independently selected from C₁₋₆ alkyl, OR^{a}, Cl, F, Br, I, ═O, —CN, NO₂, NR^{a}R^{a1}, C(O)R^{a}, C(O)OR^{a}, C(O)NR^{a}R^{a1}, R^{a}NC(O)NR^{a}R^{a1}, OC(O)NR^{a}R^{a1}, R^{a}NC(O)OR^{a}, S(O)₂NR^{a}R^{a1}, NR^{a}S(O)₂R^{a2}, NR^{a}S(O)₂NR^{a}R^{a1}, OS(O)₂NR^{a}R^{a1}, NR^{a}S(O)₂R^{a2}, S(O)ₚR^{a2}, CF₃, CF₂CF₃, CH₂F, and CHF₂;
R^{d}, at each occurrence, is independently selected from C₁₋₆ alkyl, OR^{a}, Cl, F, Br, ═O, —CN, NR^{a}R^{a1}, C(O)R^{a}, C(O)OR^{a}, C(O)NR^{a}R^{a1}, S(O)₂NR^{a}R^{a1}, S(O)ₚR^{a2}, CF₃, C₃₋₆ carbocyclic residue and a 5-6 membered heterocycle comprising: carbon atoms and 1-4 heteroatoms selected from the group consisting of N, O, and S;
R^{e}, at each occurrence, is selected from phenyl substituted with 0-2 R^{b} and biphenyl substituted with 0-2 R^{b};
R^{f}, at each occurrence, is selected from C₁₋₄ alkyl, C₃₋₆ cycloalkyl, C₁₋₅ alkoxy, phenyl substituted with 0-2 R^{b};
p, at each occurrence, is selected from 0, 1, and 2;
r, at each occurrence, is selected from 0, 1, 2, 3, and 4; and
r1, at each occurrence, is selected from 0, 1, 2, 3, and 4.
